(19) United States
(12) Patent Application Publication
(10) Pub. No.: US 2021/0054047 A1
(43) Pub. Date: Feb. 25, 2021

(54) NOVEL ANTICANCER FUSION PROTEIN AND USE THEREOF

(71) Applicants: KOREA INSTITUTE OF SCIENCE AND TECHNOLOGY, Seoul (KR); KYUNGPOOK NATIONAL UNIVERSITY INDUSTRY-ACADEMIC COOPERATION FOUNDATION, Daegu (KR)

(72) Inventors: Min-Woo KIH, Seoul (KR); Eun Jung LEE, Daegu (KR); Yoo Soo YANG, Seoul (KR); Cherl Hyun JEONG, Seoul (KR); In-San KIM, Seoul (KR); Soyoun KIM, Daegu (KR)

(73) Assignees: KOREA INSTITUTE OF SCIENCE AND TECHNOLOGY, Seoul (KR); KYUNGPOOK NATIONAL UNIVERSITY INDUSTRY-ACADEMIC COOPERATION FOUNDATION, Daegu (KR)

(21) Appl. No.: 17/013,245

(22) Filed: Sep. 4, 2020

Related U.S. Application Data
(63) Continuation-in-part of application No. PCT/KR2019/002531, filed on Mar. 5, 2019.

(30) Foreign Application Priority Data
Mar. 6, 2018 (KR) 10-2018-0026230

(51) Int. Cl. C07K 14/705; A61P 35/00

(52) U.S. Cl. CPC C07K 14/70575; A61K 38/00; A61P 35/00

(57) ABSTRACT

The present invention relates to a novel anticancer fusion protein and use thereof, and more particularly, provides a fusion protein in which a tumor necrosis factor (TNF) superfamily protein is linked to a self-assembled protein, which is capable of forming a protein nanocage by self-assembly of the self-assembled protein.

### CROSS REFERENCE TO RELATED APPLICATIONS

This application is a continuation-in-part of international patent application PCT/KR2019/002531 filed on Mar. 5, 2019, which claims priority to Korean Patent Application Ser. No. 2018-0026230 filed on Mar. 6, 2018. Both of the above applications are incorporated herein by reference in their entirety.

### SEQUENCE LISTING

The instant application contains a Sequence Listing which has been submitted electronically in ASCII format and is hereby incorporated by reference in its entirety. Said ASCII copy, created on Nov. 17, 2020, is named 122257-0113 ST25 201117 corrected.txt and is 11,657 bytes in size.

### TECHNICAL FIELD

The present invention relates to a novel anticancer fusion protein and use thereof.

### BACKGROUND

Tumor necrosis factor (TNF) ligand and receptor superfamily play an important role in the regulation of haematopoiesis, morphogenesis and immune response, and development of TNF-targeted therapeutics is currently the subject of interest. The TNF super family consists of 27 ligands and shares the extracellular TNF homology domain (THD) that induces the formation of structural features, non-covalent homo-trimers (D. W. Banner et al., Cell, (3): 431-445, 1993). Given that the endogenous TNF ligand exists as a homo-trimer and that the trimer induces the activation of downstream signaling of the receptor, the formation of the trimer structure is an important factor for its stability and biological function. Tumor necrosis factor-related apoptosis-inducing ligand (TRAIL), one of the TNF super families, is bound to TRAIL R1 (death receptor 4, DR4), TRAIL R2 (kill receptor 5, DR5), TRAIL R3 (decoy receptor 1, DcR1), TRAIL R4 (decoy receptor 2, DcR2), and osteoprotegerin, which are members of the TNFR super family 5 (A. Almasan et al., Cell Mol. Life Sci. 66(6): 981-993, 2009). Among these receptors, DR4 and DR5 contain a cytoplasmic ‘death domain’ (DD) and induce apoptosis in cells. In particular, unlike other apoptosis-inducing ligands (ie, Fas-ligand), TRAIL has been proven to be more effective in selectively inducing apoptosis of tumor cells. Based on preclinical studies, TRAIL agonists exhibit marked anti-tumor activity in various tumor types but have no or limited effect on normal cells (A. Ashkenazi et al., J. Clin. Invest. 104 (2): 155-162, 1999). Therefore, TRAIL can be considered a preferred anticancer agent due to its tumor-specific apoptotic activity.

However, recent clinical trials of TRAIL-based therapeutics (e.g., circularly permuted 8 TRAIL (CPT), AGP350, and dulanermin) exhibit no effectual anti-tumor activity toward cancer patients (Herbst et al., J. Clin. Oncol. 2010, 28(17): 2839-2846, 2010; Soria et al., J. Clin. Oncol. 28(9): 1527-33, 2010; Geng et al., Am. J. Hematol. 89(11): 1037-1042, 2014; Leng et al., Chin. J. Cancer 35: 86, 2016; Leng et al., Cancer Chemother. Pharmacol. 79(6): 1141-1149, 2017). There are some possible explanations for the failure of TRAIL-based therapeutics, but the predominant factors are 1) low apoptotic potency owing to the inability of TRAIL to form its native homo-trimeric complex structure, which is essential for activation of DR4- and DR5-mediated downstream signaling, 2) poor stability and pharmacokinetics, and 3) resistance to TRAIL-mediated apoptosis in various tumor cell types (Zhang et al., FEBS Let. 482(3): 193-199, 2000; Zhang et al., Cancer Gene Ther. 12(3): 228-237, 2005; Saraei et al., Biomed. Pharmacother. 2018, 107: 1010-1019, 2018; Geismann et al., Cell Death Dis. 5(10): e1455, 2014; Zhang et al., Gene 627: 420-427, 2017).

The present invention has been devised to solve various problems including the above-mentioned problems, thus an object of the present invention is to provide a novel anti-cancer fusion protein capable of maximizing the efficiency of cancer immunotherapy showing effective anticancer activity. However, these problems are exemplary, and the scope of the present invention is not limited thereto.

### SUMMARY OF THE INVENTION

In an aspect of the present invention, there is provided a fusion protein in which a tumor necrosis factor superfamily protein is linked to a self-assembled protein.

In another aspect of the present invention, there is provided a protein nanocage produced by self-assembly of the fusion protein.

In another aspect of the present invention, there is provided a complex protein nanocage produced by self-assembly of the fusion protein and encapsulated with an immunogenic apoptosis-inducing compound therein.

In another aspect of the present invention, there is provided a pharmaceutical composition for treating cancer comprising the protein nanocage as an active ingredient and at least one pharmaceutically acceptable carrier.

In another aspect of the present invention, there is provided a method of treating cancer in a subject comprising administering a therapeutically effective amount of the protein nanocage to the subject.

In another aspect of the present invention, there is provided a method of re-sensitizing TRAIL-resistant tumor cells to TRAIL, comprising treating the tumor cells with a complex protein nanocage as described herein with an immunogenic cell death-inducing compound or a TNF superfamily re-sensitizer encapsulated therein or with a protein nanocage as described herein and an immunogenic cell death-inducing compound or TNF superfamily re-sensitizer.

In another aspect of the present invention, there is provided a method of treating cancer in a subject suffering from TRAIL-resistant cancer, comprising administering a therapeutically effective amount of a complex protein nanocage as described herein with an immunogenic cell death-inducing compound or a TNF superfamily re-sensitizer encapsulated therein, or administering a therapeutically effective amount of a protein nanocage as described herein and an immunogenic cell death-inducing compound or TNF superfamily re-sensitizer to the subject.

### Effect of the Invention

According to an embodiment of the present invention made as described above, it is possible to implement a novel anticancer fusion protein production effect capable of maximizing the efficiency of cancer immunotherapy showing high anticancer activity by effectively inducing apoptosis of tumor cells. Of course, the scope of the present invention is not limited by these effects.

### BRIEF DESCRIPTION OF DRAWINGS

FIG. 1 is a schematic diagram showing the similarity of the trimeric structure and distance between each C-terminus of the TNF super family. The 3D protein structure was generated using RasMol (v 2.7.2) and the distance between C terminal atoms was calculated using Pymol. Blue balls indicate the C-terminus of the TNF super family ligand.

FIG. 2A-2B represent the design of a TTPN showing a natural-like trimer TRAIL complex on the ferritin surface, and show a schematic diagram showing the 3D protein structure of the extracellular domain (eco-domain) of the TRAIL trimer complex (FIG. 2A) and a schematic diagram showing the human ferritin heavy chain (hFTH-H) nanocage (PDB 2FHA) (FIG. 2B), the bold blue part represents the triaxial symmetric structure, and the blue and red spheres are the C-terminus of TRAIL and the N-terminus of the human ferritin subunit, respectively.

FIG. 3 is a diagram showing the design of a TTPN showing a natural-like trimer TRAIL complex on the ferritin surface, and a schematic plasmid vector and dimensions of the TTPN. The C-terminus of the extracellular domain of the domain TRAIL (purple) was fused to the N-terminus of the human ferritin subunit (gray) by a linker peptide (blue).

FIG. 4A4C represent an analysis of the TTPN of the present invention, and show a photograph representing the SDS-page analysis of the expressed TTPNs (FIG. 4A); a SDS-PAGE gel photograph of TTPN and wtFTN (FIG. 4B); and a Western blot analysis of purified TTPN (FIG. 4C):

Black arrow: TTPN of theoretical molecular weight (48 kDa);

M: protein marker;

IS: insoluble fraction; and

S: Soluble fraction of E. coli cell lysate.

FIG. 5A-5B represent the physicochemical properties of the TTPNs of the present invention, and show a graph representing the elution profile of size exclusion chromatography (FIG. 5A); and a series of histograms representing size distribution of nanoparticles prepared by self-assembling using dynamic light scattering (DLS) analysis of wtFTN (FIG. 5B, left) and TTPN (FIG. 5B, right).

FIG. 6 is a series of transmission electron microscopic (TEM) images of purified wtFTN (left) and TTPN (right) showing the physicochemical properties of the TTPNs of the present invention, showing a spherical cage structure.

FIG. 7 is a series of photographs showing the physicochemical properties of the TTPNs of the present invention and showing the average 2D class of TTPN processed representatively on a negative-stained electron microscope.

FIG. 8 is a series of histograms showing different expression levels of TRAIL receptors on the surface of tumor cells, HEK293T, HT29 and HepG2 cells.

FIG. 9 is a graph showing the relative mean fluorescence intensity (MFI) compared to the IgG control group showing different expression levels of TRAIL receptors on the surface of tumor cells, HEK293T, HT29 and HepG2 cells.

FIG. 10 is a series of histograms showing representative flow cytometric histogram analysis results of HEK293T, HT29, and HepG2 cells treated with 400 nM TTPN and wtFTN as an analysis of the possibility of binding TTPN to tumor cells and normal cells.

FIG. 11 is a graph showing the results of analyzing the binding potential of TTPN to tumor cells and normal cells from the flow cytometric plot of FIG. 10 to analyze the binding potential of TPPNs to cancer cells and normal cells. Data represent mean fluorescence intensity (MFI)±SEM of at least 3 independent experiments (*: p<0.05, **: p<0.01 and ***: p<0.001 vs. only cell control, ns: not significant, Student's t-test).

FIG. 12 is a histogram showing the binding specificity of TTPN to the TRAIL receptor on the tumor surface as a result of analyzing the possibility of binding TTPNs to tumor cells and normal cells. HepG2 cells sensitive to TRAIL were pre-blocked with anti-DR4, DR5, DcR1, and DcR2 antibodies and then cultured with 400 nM TTPN.

FIG. 13 is a graph representing an analysis of the binding potential of TTPNs to tumor cells and normal cells, showing the quantification of the specific binding ability calculated from the flow cytometric plots including FIGS. 11 and 12.

FIG. 14 is a series of fluorescence microscopic images representing the binding potential of TTPNs to tumor cells and normal cells. HepG2 cells treated with TTPN and wtFTN. HepG2 cells were treated with 50 nM TTPN and wtFTN, treated with anti-ferritin heavy chain and Alexa 488 antibody (green), and the nuclei were counterstained with Hoechst (blue). Scale bar: 100 μm.

FIG. 15 is a photograph showing the stability of TTPN and mTRAIL by analyzing the improved binding kinetics and affinity for DR4 and DR5 and the stability of TTPN. 15 mg/mL TTPN and wtFTN were incubated in PBS buffer for 24 hours after purification.

FIG. 16 is a graph analyzing the results of monitoring the stability of TTPN and mTRAIL (10 mg/mL) for 1 month as an analysis of improved binding kinetics and affinity for DR4 and DR5 and the stability of TTPN. Data represent the mean±SEM of at least 3 independent experiments (*: p<0.05, **: p<0.01, and ***: p<0.001 vs mTRAIL, uniformity with Tukey post-hoc test. ANOVA analysis).

FIG. 17 is a graph representing cell viability according to treatment with TTPN and mTRAIL. HepG2 cells were cultured for 24 hours in the presence of different concentrations of mTRAIL and TTPN, and analyzed with a cell counting kit (CCK-8).

FIG. 18 is a graph analyzing the apoptotic activity of TTPN and mTRAIL against HEK293T normal cells in vitro.

FIG. 19 is a series of histograms representing flow cytometry analysis of TRAIL-mediated apoptosis of HepG2 cells as an analysis of in vitro TTPN-mediated apoptosis on TRAIL-sensitive-HepG2 cells. Cells were incubated for 24 hours in the presence of different concentrations of mTRAIL and TTPN and analyzed by Annexin V/PI double staining.

FIG. 20 is a graph showing the proportion of Annexin-V positive cells among TRAIL-sensitive HepG2 cells.

FIG. 21 is a graph showing the analysis of in vitro TTPN-mediated apoptosis of TRAIL-sensitive HepG2 cells, and analysis of the proportion of Annexin-V positive cells calculated in the flow cytometry plot including FIG. 18. Data represent the mean±SEM of at least 3 independent experiments (*: p<0.05, **: p<0.01 and ***: p<0.001 vs. buffer control, one-way ANOVA analysis along with Tukey's post-test).

FIG. 22A-22D are described herein. FIG. 22A is a schematic diagram of the process for preparing DOX-TTPNs based on the 3D protein structure of the ecto-domain of the TRAIL trimeric complex (PDB 1DG6) and human ferritin heavy chain (hFTN-H) nanocages (PDB 2FHA); FIG. 22B is a graph representing fluorescence intensity of TTPN (0.3 mg/ml) before and after encapsulation of DOX with excitation and emission at 470 and 565˜650 nm, respectively; FIG. 22C is a size-exclusion chromatography elution profile of DOX-TTPNs showing successful encapsulation of DOX into TTPNs (TTPN absorbance λ, 280 nm; DOX absorbance λ, 480 nm); and FIG. 22D is a histogram representing DLS analysis of DOX-TTPNs showing no significant difference in size compared with empty TTPNs.

FIG. 23 is a graph showing in vitro stability of TTPNs and DOX-TTPNs.

FIG. 24A-24C represent a result of observing after intravenous injection of wtFTN, mTRAIL, and TTPN labeled with Cy5.5 into a HepG2 tumor bearing mouse model in order to analyze ex vivo delivery efficiency of intravenous TTPNs to the tumor, and show an ex vivo near-infrared fluorescence (NIRF) image of excised major organs including liver, lung, spleen, kidney, heart, intestine and tumor 24 hours after intravenous injection of wtFTN, mTRAIL and TTPN, respectively (FIG. 24A); a series of ex vivo tumor near-infrared fluorescence (NIRF) images (FIG. 24B); and a graph representing the quantitative near-infrared fluorescence intensity of the excised tumor indicated in B are shown (FIG. 24C).

FIG. 25 is a graph analyzing the anti-tumor effect of TTPN-mediated apoptosis on tumor growth of HepG2 tumor-bearing mice. TTPN-, wtFTN-, mTRAIL-, and buffer-treated mice were analyzed for tumor growth rates. After tumor volume reached ˜80-100 mm³, mice were treated with TTPN (23 mg/kg), wtFTN (10 mg/kg, corresponding to molecules of ferritin within 23 mg/kg of TTPN), mTRAIL (12 mg/kg, corresponding to molecules of TRAILs within 23 mg/kg of TTPN) or buffer (control) was administered 6 times every 2 times via intravenous inoculation (n=6 mice/group).

FIG. 26 is a representative picture of TTPN- and buffer-treated mice 25 days after tumor inoculation (HepG2 tumor on the left side, scale bar=1 cm).

FIG. 27 is a photograph showing a tumor excised at the end of the experiment in FIG. 25.

FIG. 28 is a graph representing the weight of the excised tumors at the end of the experiment of FIG. 25.

FIG. 29 is a series of representative fluorescence microscopic images showing apoptosis in tumor sections of TTPN and wtFTN treated mice using TUNEL analysis.

FIG. 30 is a graph showing the results of quantitative analysis of apoptotic cells in tumor tissue slices analyzed by fluorescence images including FIG. 29 by ImageJ software. Data represent mean±SEM (*: p<0.05, **: p<0.01, and ***: p<0.001 vs buffer control, NS is not significant, one-way ANOVA analysis with Tukey's post-test).

FIG. 31 is a representative series of SPR sensograms for mTRAIL and TTPN bound to immobilized DR4 and DR5, respectively. The concentration of injected analyte is displayed.

FIG. 32 is a series of fluorescence microscopic images representing time-course tracking of DOX-TTPNs within HT29 tumor cells. Cells were incubated with 40 nM Cy5-conjugated DOX-TTPNs and analyzed by confocal microscopy at different time points (panels A-D). Representative images showing the distribution of Cy5-TTPNs (green) and DOX (red) at 0 min (panel A), 30 min (panel B), 1 h (panel C), and 2 h (panel C). Nuclei were counterstained with DAPI (blue). Scale bars: 25 μm.

FIG. 33A-33C are described herein. FIG. 33A is a graph representing cell viability of HT29 cells, determined using CCK assays, after treatment with equal molar concentrations of DOX-TTPNs, DOX-FTNs, free DOX (equivalent to the DOX dose in DOX-TTPNs), or empty TTPNs (equivalent to the number of moles of TRAIL in DOX-TTPNs) for 24 h; FIG. 33B is a graph representing cell viability of MCF7 cells, determined using CCK assays, after treatment with equal molar concentrations of DOX-TTPNs, DOX-FTNs, free DOX (equivalent to the DOX dose in DOX-TTPNs), or empty TTPNs (equivalent to the number of moles of TRAIL in DOX-TTPNs) for 24 h; and FIG. 33C is a series of representative flow cytometry histograms showing DOX-TTPN-induced apoptosis of HT29 cells at low molar concentrations of DOX-TTPNs. Cells were incubated with the indicated preparation for 24 h, and then analyzed for staining of the early apoptosis marker, annexin V (red, DOX-TTPNs; blue, empty TTPNs; gray, free DOX; black, buffer) Data represent means±SEM (***P<0.001, compared to buffer control with the exception of over-proliferation; Student's t test).

FIG. 34A-34C represent in vitro re-sensitization effect of DOX-TTPNs in HT29 cells: analysis of the levels of TRAIL-mediated, apoptosis-related proteins, shows a representative Western blot analysis of the pro-apoptotic proteins, cleaved caspase-8 (Cl-caspase-8) and Cl-caspase-3; apoptosis-initiator, caspase-8; and anti-apoptotic proteins, Bax-xL, c-FLIPL/S, XIAP. Cells were treated with DOX-TTPNs, empty TTPNs, free DOX, or buffer for 24 h and cell lysates were examined by Western blotting (FIG. 34A); a representative Western blot analysis showing DR5 levels in time-course tracking experiments (FIG. 34B) and a schematic diagram showing intracellular delivery of DOX through the DR5-mediated endocytosis pathway (FIG. 34C).

FIG. 35A-35D are described herein. FIG. 35A is a graph representing tumor growth rate in HT29 tumor-bearing mice treated with DOX-TTPNs, DOX-FTNs, empty TTPNs, free DOX or buffer. After tumor volumes reached 80-100 mm³, mice were treated with DOX-TTPNs (30 mg/kg; equivalent to a DOX dose of 0.4 mg/kg), DOX-FTNs (15 mg/kg; equivalent to the number of moles of ferritin in a dose of TTPNs), empty TTPNs (30 mg/kg; equivalent to the number of moles of TRAIL in a dose of TTPNs), DOX (0.4 mg/kg), or buffer (control) five times every 2 d via intravenous injection (n=6 mice/group); FIG. 35B is a series of representative pictures of DOX-TTPN- and buffer-treated mice on day 25 post-tumor challenge. (HT29 tumors on left flank); FIG. 35C is a series of representative fluorescence microscopic images of apoptotic cells in TUNEL-stained tumor sections from mice treated with DOXTTPNs, DOX-FTNs, empty TTPNs, free DOX or buffer; and FIG. 35D is a graph representing quantification of apoptotic cells in tumor sections, determined by analysis of fluorescence images in C using ImageJ software. Data represent means±SEM (*P<0.05, compared with buffer control; Student's t test). Scale bars: 50 μm.

### DETAILED DESCRIPTION OF THE INVENTION

### Definition of Terms

The term “nanocage” as used herein refers to hollow nanoparticles, which include inorganic nanocages and organic nanocages. Inorganic nanocage is hollow and porous gold nanoparticles produced by reacting silver nanoparticles with chloroauric acid (HAuCl₄) in boiling water and organic nanoparticles include protein nanocages, which are nanocages produced by self-assembly of self-assembled proteins such as ferritin.

The term “complex nanocage” as used herein refers to a nanocage in which a specific material is loaded in an empty space of the nanocage. For example, when doxorubicin, an anticancer agent, is loaded inside a protein nanocage composed of ferritin heavy-chain protein, it becomes a doxorubicin complex protein nanocage. “Doxorubicin complex nanocage” may be used herein as the same meaning as “doxorubicin-loaded nanocage”, “doxorubicin complex protein nanocage”, or “doxorubicin-loaded protein nanocage”.

The term “tumor necrosis factor-related apoptosis-inducing ligand (TRAIL)” as used herein refers to a protein that functions as a ligand that induces apoptosis process called apoptosis. TRAIL is a type of cytokine produced and secreted by most normal tissue cells. It usually induces apoptosis in tumor cells by binding to specific tumor receptors.

The term “trimeric TRAIL-presenting nanocage” (hereinafter referred to as “TTPN”) as used herein refers to a protein nanocage that presents TRAIL as a natural-like homo-trimeric structure on the surface. The TTPN is designed and manufactured by the inventors.

The term “TNF superfamily” as used herein refers to a super family of cytokines that can induce apoptosis. The tumor necrosis factor (TNF, formerly known as TNFα) is the most well-known member of this class, and TNF is a monocyte-derived cellular toxin associated with tumor regression, septic shock and cachexia.

The term “TNF superfamily re-sensitizer” as used herein refers to a compound or composition that plays a role of restoring sensitivity to the TNF superfamily of tumor cells resistant to the TNF superfamily.

The term “immunogenic cell death” refers to a type of cell death caused by cell growth inhibitors such as anthracyclines, taxan-based chemotherapeutic agents, oxaliplatin and bortezomib, radiotherapy or photodynamic therapy. Unlike general apoptosis, the immunogenic cell death can induce an effective anticancer immune response through activation of dendritic cells and activation of specific T cell responses thereby. A substance inducing immunogenic cell death is called an immunogenic cell death inducer or an immunogenic cell death-inducing compound. The immunogenic cell death and the immunogenic cell death-inducing compounds are well described in a prior art (Kroemer et al., Annu. Rev. Immunol., 31: 51-72, 2013). This document is incorporated herein by reference in its entirety.

The term “therapeutically effective amount” as used herein refers to an amount sufficient to significantly ameliorate the symptoms of a disease when administered to a subject in need of treatment. The “therapeutically effective amount” can be appropriately selected according to the cell or individual selected by a person skilled in the art. “Therapeutically effective amount” can be determined according to the degree of disease, age, weight, health, sex, susceptibility to drugs, administration time, administration route and excretion rate, treatment period, methods of preparing composition used, and other factors well-known in the art including drugs used in combination with. The effective amount may be about 0.5 μg to about 2 g, about 1 μg to about 1 g, about 10 μg to about 500 mg, about 100 μg to about 100 mg, or about 1 mg to about 50 mg per composition.

### BEST MODES

In an aspect of the present invention, there is provided a fusion protein in which a tumor necrosis factor (TNF) superfamily protein is linked to a self-assembled protein.

With regard to the fusion protein, the TNF superfamily protein may be TRAIL, CD40L (CD40 ligand), OX40L (OX40 ligand), FasL (Fas ligand), LIGHT (tumor necrosis factor superfamily member 14), APRIL (A proliferation-inducing ligand), TNF-α (tumor necrosis factor alpha), TNF-β (tumor necrosis factor-beta), VEGI (vascular endothelial growth inhibitor), BAFF (B-cell activating factor), RANKL (receptor activator of nuclear factor kappa-B ligand), LT (lymphotoxin)α/LT (lymphotoxin)β, TWEAK (TNF-related weak inducer of apoptosis), CD30L (CD30 ligand), 4-1BBL (4-1BB ligand), GITRL (glucocorticoid-induced TNF-related ligand), or EDA-A (ectodysplasin A).

In an embodiment of the present invention, TRAIL was used, but other TNF superfamily proteins forming homologous trimers may also be used by adjusting the type and size of the linker according to the size.

With regard to the fusion protein, the self-assembled protein may be a small heat shock protein (sHsp), ferritin, vault, P6HRC1-SAPN, M2e-SAPN, MPER-SAPN, or a virus or bacteriophage capsid protein. The ferritin may be a ferritin heavy chain protein or a ferritin light chain protein. In one embodiment of the present invention, the ferritin heavy chain protein was used as the self-assembled protein, but other self-assembled proteins capable of forming a spherical nanocage by self-assembly thereof may also be used. In this case, by controlling the type and size of the linker according to the size of the self-assembled protein, it is possible to maintain the triaxial symmetric structure of the prepared protein nanocage.

Accordingly, in an aspect of the present invention, there is provided a fusion protein in which TRAIL is linked to a ferritin protein.

With regard to the fusion protein, the TRAIL may be linked to the N-terminus or C-terminus of the ferritin protein, and may further include a linker peptide between the ferritin protein and TRAIL.

with regard to the fusion protein, the length of the linker peptide may be 2 to 50 aa, and the linker peptide may be selected from the group consisting of A(EAAAK)₄ALEA(EAAAK)₄A (SEQ ID NO: 4), (G₄S)ₙ (where n is an integer from 1 to 10), (GS)ₙ (where n is an integer from 1 to 10), (GSSGGS)ₙ (SEQ ID NO: 15, where n is an integer from 1 to 10), KESGSVSSEQLAQFRSLD (SEQ ID NO: 16), EGKSSGSGSESKST (SEQ ID NO: 17), GSAGSAAGSGEF (SEQ ID NO: 18), (EAAAK)ₙ (SEQ ID NO: 19, where n is an integer from 1 to 10), CRRRRRREAEAC (SEQ ID NO: 20), GGGGGGGG (SEQ ID NO: 21), GGGGGG (SEQ ID NO: 22), AEAAAKEAAAAKA (SEQ ID NO: 23), PAPAP (SEQ ID NO: 24), (Ala-Pro)ₙ (where n is an integer from 1 to 10), VSQTSKLTRAETVFPDV (SEQ ID NO: 25), PLGLWA (SEQ ID NO: 26), TRHRQPRGWE (SEQ ID NO: 27), AGNRVRRSVG (SEQ ID NO: 28), RRRRRRRR (SEQ ID NO: 29), and GSSGGSGSSGGSGGGDEADGSRGSQKAGVDE (SEQ ID NO: 30). As described above, the length of the linker may be appropriately adjusted according to the type and size of the self-assembled protein and/or the TNF superfamily protein.

In another aspect of the present invention, there is provided a protein nanocage produced by self-assembly of the fusion protein.

In another aspect of the present invention, there is provided a complex protein nanocage produced by self-assembly of the fusion protein and encapsulation of an immunogenic cell death-inducing compound or a TNF superfamily re-sensitizer therein.

With regard to the complex protein nanocage, the immunogenic cell death-inducing compound may be an anti-EGFR antibody, a BK channel agonist, bortezomib, the combination of cardiac glycoside and a non-immunogenic apoptosis inducer, cyclophosphamides, the combination of GADD34/PP1 inhibitor and mitomycin, LV-tSMAC, Measles virus, or oxaliplatin.

With regard to the complex protein nanocage, the TNF superfamily re-sensitizer may be doxorubicin, cisplatin, gemcitabine, oxaliplatin, irinotecan, camptothecin, celecoxib, curcumin, cinobufotalin, berberine, LY294002, wortmannin, ABT-737, HA14-1, or p53 reactivation or induction of massive apoptosis (PRIMA-1).

In another aspect of the present invention, there is provided a pharmaceutical composition for treating cancer comprising the protein nanocage or the complex protein nanocage as described herein as an active ingredient and at least one pharmaceutically acceptable carrier.

The pharmaceutical composition for cancer treatment may further include an immunogenic cell death-inducing compound or a TNF superfamily re-sensitizer. The immunogenic cell death-inducing compound may be an anti-EGFR antibody, a BK channel agonist, bortezomib, the combination of cardiac glycoside and a non-immunogenic apoptosis inducer, cyclophosphamides, the combination of GADD34/PP1 inhibitor and mitomycin, LV-tSMAC, Measles virus, or oxaliplatin. The TNF superfamily re-sensitizer may be doxorubicin, cisplatin, gemcitabine, oxaliplatin, irinotecan, camptothecin, celecoxib, curcumin, cinobufotalin, berberine, LY294002, wortmannin, ABT-737, HA14-1, or p53 reactivation or induction of massive apoptosis (PRIMA-1).

In another aspect of the present invention, there is provided a method of treating cancer in a subject comprising administering a therapeutically effective amount of the protein nanocage or the complex protein nanocage as described herein to the subject.

In another aspect of the present invention, there is provided a method of re-sensitizing TRAIL-resistant tumor cells to TRAIL, comprising treating the tumor cells with a complex protein nanocage as described herein, comprising a protein nanocage produced by self-assembly of a fusion protein as described herein and an immunogenic cell death-inducing compound or a TNF superfamily re-sensitizer encapsulated therein, or with a protein nanocage as described herein and an immunogenic cell death-inducing compound or TNF superfamily re-sensitizer.

In another aspect of the present invention, there is provided a method of treating cancer in a subject suffering from TRAIL-resistant cancer, comprising administering a therapeutically effective amount of a complex protein nanocage as described herein, comprising a protein nanocage produced by self-assembly of a fusion protein as described herein and an immunogenic cell death-inducing compound or a TNF superfamily re-sensitizer encapsulated therein, or administering a therapeutically effective amount of a protein nanocage as described herein and an immunogenic cell death-inducing compound or TNF superfamily re-sensitizer to the subject.

The therapeutically effective amount may vary depending on the type of the subject's (patient's) affected area, the application site, the number of treatments, the treatment time, the formulation, the subject's (patient's) condition, the type of adjuvant, and the like. The amount used is not particularly limited, but may be 0.01 μg/kg/day to 10 mg/kg/day. The daily dose may be administered once a day, or divided into 2-3 times a day at appropriate intervals, or intermittently administered at intervals of several days.

The active agent of the present invention may be present in the composition in an amount of 0.1-100% by weight based on the total weight of the composition, which may further include suitable carriers, excipients, and diluents commonly used in the preparation of pharmaceutical compositions. In addition, solid or liquid additives for preparation may be used in the preparation of pharmaceutical compositions. The additive for formulation may be either organic or inorganic. Examples of excipients include lactose, sucrose, sucrose, glucose, cornstarch, starch, talc, sorbit, crystalline cellulose, dextrin, kaolin, calcium carbonate, and silicon dioxide. As a binder, for example, polyvinyl alcohol, polyvinyl ether, ethyl cellulose, methyl cellulose, gum arabic, tragacanth, gelatin, shellac, hydroxypropyl cellulose, hydroxypropyl methyl cellulose, calcium citrate, dextrin and pectin. Examples of the lubricant include magnesium stearate, talc, polyethylene glycol, silica, and hydrogenated vegetable oil. Any colorant that is permitted to be added to pharmaceuticals can be used. These tablets and granules can be appropriately coated with a sugar coat, gelatin coating, or other necessary. In addition, preservatives, antioxidants, and the like may be added as necessary.

The pharmaceutical composition of the present invention may be prepared in any formulation conventionally prepared in the art (for example, Remington's Pharmaceutical Science, the latest edition; Mack Publishing Company, Easton Pa.), and the form of the formulation is not particularly limited. Exemplary formulations are described in Remington's Pharmaceutical Science, 15^{th} Edition, 1975, Mack Publishing Company, Easton, Pa. 18042 (Chapter 87: Blaug, Seymour). These and other such formulation are well known for all pharmaceutical chemistry.

The pharmaceutical composition of the present invention may be administered orally or parenterally, preferably wherein parenteral administration is by intravenous injection, subcutaneous injection, intracerebroventricular injection, intracerebrospinal fluid injection, intramuscular injection or intraperitoneal injection.

Tumor necrosis factor-related apoptosis-inducing ligand (TRAIL), which is one of the TNF superfamily, may bind to a TNFR superfamily member including TRAIL R1 (death receptor 4, DR4), TRAIL R2 (death receptor 5, DR5), TRAIL R3 (decoy receptor 1, DcR1), TRAIL R4 (decoy receptor 2, DcR2), and osteoprotegerin. Among these receptors, DR4 and DR5 contain a cytoplasmic ‘death domain’ (DD) and induce apoptosis of cells. In particular, unlike other apoptosis-inducing ligands (i.e., Fas-ligands), TRAIL has proven to be more effective in selectively inducing apoptosis of tumor cells. Based on preclinical studies, TRAIL agonists showed remarkable anti-tumor activity in various tumor types, but no or limited effects on normal cells. Thus, TRAIL can be considered a desirable anticancer agent due to its tumor-specific apoptotic activity. Like other members of the TNF superfamily, endogenous TRAIL exists as a homo-trimeric complex that is critical for stability, solubility and bioactivity. According to recent studies, several types of trimer preparations of recombinant TRAIL have been reported to improve biological properties such as stability, delivery and cytotoxic activity against tumors (i.e., FLAG and its tag-mediated crosslinking; linking it to the Fc portion of IgG; fusion of trimerization domains such as leucine zippers or isoleucine zippers; conjugated to nanoparticles; stabilization of trimers with cations, etc.) (D. Merino et al., Expert Opin. Ther. Targets. 11: 1299-1314, 2007).

When expressed in the form of a recombinant protein by linking a TNF superfamily protein such as TRAIL to a self-assembled protein such as a ferritin heavy chain with a linker peptide, it was confirmed that a protein nanocage could present the TNF superfamily ligand as active homo-trimers on the surface of the protein nanocage, by adjusting the length of the linker peptide. Further, it was confirmed that the protein nanocage alone or a complex protein nanocage prepared by encapsulating an immunogenic cell death-inducing compound such as doxorubicin in the protein nanocage can promote the death of tumor cells in cancer model mice when administrated to the cancer model mice. In addition, it was confirmed that the complex protein nanocage comprising the protein nanocage and an immunogenic cell death inducer encapsulated therein according to an embodiment of the present invention restored sensitivity of TRAIL-resistant cancer cells to TRAIL. Moreover, the effect of the present invention was achieved even with a significantly lower dose (even 0.4 mg/kg) of doxorubin compared to conventional combination TRAIL and anticancer drug therapy using, e.g., 1.5 to 7 mg/kg of anticancer drug. Thus, the complex protein nanocage according to an embodiment of the present invention showed an unexpected prominent and enhanced effect over the prior art.

Hereinafter, the present invention will be described in more detail through examples. However, the present invention is not limited to the examples disclosed below, but can be implemented in various different forms, and the following embodiments are intended to complete the disclosure of the present invention, and fully inform the scope of the invention to those of ordinary skill in the art.

### General Methods

### Design and Biosynthesis of TTPNs

For the generation of wtFTN, mTRAIL and TTPN, gene clones were prepared through polymerase chain reaction (PCR) amplification using appropriate primers; i) N-NdeI-6×His tag-(hFTH)-HindIII-C; ii) N-NdeI-(TRAIL 95-281)-BamHI; iii) N-NdeI-(TRAIL₉₅₋₂₈₁)-BamHI-linker-XhoI-(hFTH)-HindIII-C; polynucleotides encoding hFTH (SEQ ID NO: 5) and TRAIL (SEQ ID NO: 1), respectively, were cloned using a cDNA clone (Sino Biological Inc., China), and a polynucleotide encoding a linker peptide A(EAAAK)₄ALEA(EAAAK)₄A (SEQ ID NO: 3) was cloned through expansion PCR amplification using appropriate primers. The gene clone was ligated with the vector for construction of the expression vector (pET28a for wtFTN and TTPN, pT7 for wtFTN): pT7-wtFTN, pET28a-mTRAIL, pET28a-TTPN. After complete sequencing, the expression vector was transformed with E. coli strain BL21(DE3) [F-ompT hsdSB(rB-mB-)] with ampicillin (for pT7) or kanamycin (for pET28a).

Cells transformed with wtFTN, mTRAIL and TTPN constructs were grown to OD₆₀₀=0.6 level at 37° C. in LB medium containing appropriate antibiotics (ampicillin for wtFTN, mTRAIL, and kanamycin for TTPN) and protein expression was induced with 0.5 mM IPTG, and grown at 20° C. for 16 hours. After growth, the cells were obtained by centrifugation, and the pellet was resuspended in a lysis buffer (0.5 M Tris-HCl (pH 7.4), 150 mM NaCl, 10 mM imidazole, 1 mM PMSF), and homogenized with a sonicator. The recombinant protein was purified through a Ni-NTA chromatography step, and after synthesis and purification, the soluble protein was stored in a storage buffer (0.5 M Tris-HCl, 150 mM NaCl, pH 7.4).

TABLE 1
Primers used for the present invention

| Primer | nucleotide sequence (5′->3′) | SEQ ID NO: |
|---|---|---|
| wtFTN F | CATATGCATCACCATCACCATCACACGACC | 7 |
| wtFTN R | AAGCTTTTAGCTTTCATTATCACT | 8 |
| mTRAIL F | CATATGACCTCTGAGGAAACCATT | 9 |
| mTRAIL R | GGATCCTTAGCCAACTAAAAAGGCCCC | 10 |
| TTPN 1 | CATATGACCTCTGAGGAAACCATT | 11 |
| TTPN 2 | CTCGAGACGACCGCGTCCACCTCGCAG | 12 |
| TTPN 3 | GGATCCGCCAACTAAAAAGGCCCCAAA | 13 |
| TTPN 4 | AAGCTTTTAGCTTTCATTATCACT | 14 |

### Design and Synthesis of DOX-TTPN

DOX (Sigma-Aldrich) was pre-incubated with CuCl₂ at a 2:1 molecular ratio of DOX to Cu²⁺ at room temperature for 20 min, as described previously. TTPNs were incubated with Cu(II)-DOX solution (1:200 molecular ratio of TTPNs to DOX) at 4° C. overnight. Free DOX was removed by dialysis and the encapsulation of DOX into TTPNs was analyzed by SEC using a Superdex 200 column (GE Healthcare). The amount of loaded DOX in TTPNs was determined by measuring fluorescence intensity of DOX using a DS-11 FX+ Fluorometer (DeNovix). After the disassembly process of DOX-TTPNs by mixing with 0.5 N HCl for 1 hour, the number of DOX molecules was quantified relatively using a standard curve of DOX fluorescence intensity.

### Analysis of Physicochemical Properties of TTPN

Size Exclusion Chromatography (SEC) and Dynamic Light Scattering (DLS) Analysis

Purified protein samples (Superdex 200 10/300, GL column) were applied to a size exclusion chromatography analyzer (SEC, Akta 100 purifier) to determine purity and molecular weight. The elution profile of TTPN was monitored by measuring the absorbance at 280 nm compared to wtFTN. The hydrodynamic sizes of TTPN and wtFTN were analyzed by dynamic light scattering analysis (DLS) and zeta potential measured using Zetasizer Nano ZS (Malvern Instruments, Ltd., UK).

Transmission Electron Microscopy (TEM) and Dynamic Light Scattering Analysis

TEM analyses of TTPN and wtFTN were imaged by bio transmission electron microscopy (Hitachi). TTPN and wtFTN (0.1 mg/ml) were placed on carbon film 200 copper grid (Electron Microscopy Science) and negatively stained using 2% ammonium molybdate. The hydrodynamic sizes of TTPN-DOX and TTPN were analyzed by dynamic light scattering (DLS) using a Zetasizer Nano ZS system (Malvern Instruments, Ltd., UK), as previously described (Zidi et al., Med. Oncol. 2010, 27(2): 185-198, 2010).

Analysis of In Vitro Stability of DOX-TTPN and TTPN

The stability of TTPN, DOX-TTPN and mTRAIL was investigated by measuring changes of concentration in a suitable buffer solution. DOX-TTPN and TTPN (8 mg/mL) in microcentrifuge tubes were monitored for one month at 4° C. At predetermined times, aggregated proteins were collected at 13,000 rpm for 10 minutes, and the concentration of soluble DOX-TTPN and TTPN in the supernatant was measured using absorbance in UV280.

### In Vitro Binding Property of TTPNs

### Tumor Cell Culture

Human colorectal adenocarcinoma cell line (HT29), hepatocellular carcinoma cell line (HepG2), and breast cancer cell line (MCF7) were provided from the Korean Cell Line Bank. The tumor cells were cultured in RPMI-1640 media supplemented with 10% fetal bovine serum (FBS) and 1% antibiotic antimycotic (AA).

### Analysis of Cell-Binding of TTPNs

TRAIL receptor expression was evaluated on various cell surfaces. Specifically, HepG2, HT29, and HEK293T cells (2×10⁵) were cultured with four types of anti-human TRAIL receptor antibodies (R&D system, MAB347, MAB6311, MAB6301, MAB633). For cell binding analysis to the TRAIL receptor, various cells were treated with 400 nM TTPN or wtFTN in a buffer solution at 4° C. for 20 minutes, followed by treatment with anti-ferritin antibody (ab65080) and anti-rabbit Alexa fluor 488 secondary antibody (Jackson Immunoresearch). Nanocage bound cells were detected with an Accuri™ C6 flow cytometer (BD Biosciences) and analyzed using FlowJo_V10 software (FlowJo). The binding specificity of TTPN to TRAIL receptor was analyzed by blocking experiments by pre-culture of anti-human TRAIL receptor antibodies and cells at 4° C. for 20 minutes. In addition, for fluorescence microscopy analysis, HepG2 cells were plated on 35-mm glass-bottom dishes, treated with 50 nM of TTPN or wtFTN buffer, and cultured with the same antibody as described above. Thereafter, the cells were fixed with 4% paraformaldehyde and stained with Hoechst 33258 before analysis for cell binding detection in a fluorescence microscope (Nikon Eclipse Ti, Nikon). Data were analyzed using LAS AF Lite software (Leica).

Surface Plasmon Resornace (SPR) Analysis

Binding experiments of TTPN and mTRAIL to TRAIL receptors DR4 and DR5 were analyzed at 25° C. using a surface plasmon resonance apparatus (SR7500 DC, Reichert Inc., NY, USA). DR4 and DR5-fc chimeric proteins (R&D systems 347-DR-100, 631-T2-100) were immobilized on the surface of the Planar Protein A sensor chip (Reichert, 13206069) and the receptors were coated at a level of 300 to 500 resonance units. SPR kinetic titrations were performed by adding 250 μl of TTPN and mTRAIL with different concentrations in the range of 1.56 to 400 nM and 0.1 to 25.6 μM, respectively, increasing by four times. Each analyte was run at 50 μL/min using running and sample buffer (0.5 M Tris-HCl (pH 7.4), 150 mM NaCl, 0.005% Tween 20), and binding of the ligand to the receptor was performed and monitored in real time. For titration sensorgrams, a simple 1:1 Langmuir interaction model (A+B↔AB) was applied using the data analysis program Scrubber 2.0 (BioLogic Software, Australia, and KaleidaGraph Software, Australia) and CLAMP software.

Time-Corse Tracking Analysis of DOX-TTPN

The efficiency of DOX-TTPN intracellular delivery was investigated using in vitro time-course tracking studies of Cy5.5-conjugated DOX-TTPNs. For conjugation of Cy5.5, Cy5.5-NHS were incubated with DOX-TTPNs at 24:1 molar ratio, followed by removal of free Cy5.5 using an Amicon Ultra Centrifugal Filter (Millipore), as previously described (Kih et al., Biomaterials 180: 67-77, 2018). After collecting a to sample, HT29 cells were incubated with 40 nM Cy5-conjugated DOX-TTPNs for 30 minutes, 1 hour and 2 hours; after cell fixation, nuclei were stained with DAPI and images were analyzed by confocal fluorescence microscopy (Leica).

### In Vitro Apoptosis Analysis of TTPN

Cell Viability Analysis

Cytotoxicity analysis was performed using mTRAIL, TTPN and wtFTN as a control. Specifically, HepG2 cells or HEK293T cells were plated on a 96-well plate, and mTRAIL, TTPN, and wtFTN were added to each well the next day at an increased concentration from 0 to 32 μM. After culturing for 24 hours, cell viability was measured using a cell counting kit (CCK)-8 assay (Dojindo Molecular Technologies, Gaithersburg, Md.). The plate was then read with an absorbance microplate reader (Spectramax 340, Molecular Devices Corporation) at a wavelength of 450 nm and 50% effective by regression analysis using SigmaPlot software (Systat Software, Inc., San Jose, Calif.). The concentration value was calculated.

In Vitro Apoptosis Analysis

HepG2, HT29, or MCF7 cells (1.5×10⁴) were cultured in a 96-well plate for 1 day, after which DOX-TTPNs, DOX-FTNs, free DOX, empty TTPNs, or buffer was added to each well. After 24 hours, cell viability was measured using a CCK-8 assay (Dojindo Molecular Technologies) according to the manufacturer's instructions. For analysis of early apoptosis, HT29 cells in 6-18 well culture plate were treated with DOX-TTPNs, free DOX, empty TTPNs, or buffer for 24 hours. Thereafter, cells were incubated with Alexa Fluor 488-conjugated annexin V (Invitrogen) 20 for 15 minutes, then analyzed using an Accuri™ C6 flow cytometer (BD Biosciences) and FlowJo 21 v10 software.

Animals

Male BALB/c nude mice (6-7 weeks old; 20 g) were purchased from Orient Bio Inc. (Seongnam, Korea). All mice were used at 7-9 weeks old after a period of stabilization. Mice were grouped randomly before xenograft, and all animals used in experiments were analyzed. All experiments using live animals were performed in compliance with the relevant laws and institutional guidelines of Korea Institute of Science and Technology (KIST) with the approval of relevant institutional committees.

In Vivo Tumor Targeting and Biodistribution of TTPN

The delivery efficiency of TTPN to the tumor microenvironment was investigated by performing an in vivo biodistribution study (n=4 mice/group) of Cy5.5-labeled TTPN using the eXplore Optix System (Advanced Research Technologies Inc., USA). Particularly, for Cy5.5 binding, TTPN, wtFTN, and mTRAIL were cultured with Cy5.5-Maleimide (Bioacts, Korea) at a molar ratio of 1:24 in a sample buffer, and then cultured at 4° C. for 16 hours. Free-Cy5.5 was separated by ultrafiltration (Amicon Ultra 100 K, Millipore), and the fluorescence intensity of the Cy5.5-labeled protein was measured using a fluorescence microplate reader (Infinite M200 Pro, TECAN, Austria). Thereafter, the same concentration and fluorescence intensity of Cy5.5-labeled TTPN, wtFTN or mTRAIL were injected intravenously into BALB/c nude mice bearing HepG2 tumors via tail vein. The fluorescence intensity of all samples was adjusted to the same value based on the data obtained using a fluorescence microplate reader. To analyze the fluorescence intensity of tumors, the Analysis Workstation software (Advanced Research Technologies Inc.) was used to calculate total photons per centimeter per steradian (p/s/cm²/sr) in the region of interest (ROI). At 24 hours post-injection, the mice were sacrificed, and tumors and major organs including liver, lung, spleen, kidney and heart were excised and analyzed in the same manner as described above.

In Vivo Antitumor Efficacy and TUNEL Analysis

In the evaluation of the anti-tumor effect of the present invention, BALB/c nude male mice (6-7 weeks old) were used as an animal model. Particularly, HT29 cells (5×10⁶) and HepG2 cells (5×10⁶) were subcutaneously inoculated into the left dorsal flank of BALB/c nude mice, respectively. After a volume of tumors reached ˜80-100 mm³, mice were randomly divided into the 14 following five treatment groups (6 mice/group); DOX-TTPNs, DOX-FTNs, free DOX, empty TTPNs, and buffer. Mice were intravenously injected five times, once every 2 days, and volume of tumors was determined as 1/2(Length×Width²). Apoptotic cell death in tumor tissues was analyzed using TUNEL staining (In situ Cell Death Detection kit; Roche), and cells were stained with DAPI (4′,6-diamidino-2-phenylindole), as previously described (Kih et al., Biomaterials 180: 67-77, 2018). Apoptotic cells were visualized under a Nikon Eclipse Ti microscope (Nikon) and quantified as the number of TUNEL-positive cells per total number of cells using ImageJ software. After 21 days from injecting tumor cells, tumor tissues were excised from the experimental mice, and cryo-sections (3.5 μm) were fixed with 10% neutral buffered formalin and paraffin-embedded tissue blocks.

Western Blot Analysis

After incubating HT29 cells with 50 nM DOX-TTPNs, DOX-FTNs, free DOX, or buffer for 24 hours, cells were lysed with radioimmunoprecipitation assay (RIPA) buffer (Cell Signaling Technology) and the concentration of proteins was measured using a DC protein assay kit (Bio-Rad). Equal amounts of proteins (30 μg) were resolved by SDS-PAGE and transferred to nitrocellulose membranes. After blocking with 5% skim milk in Tris-buffered saline containing 0.1% Tween-20 (TBST), membranes were incubated first with anti-Cl-PARP, anti-Cl-caspase-6, anti-cFLIP, anti-Bcl-xL, anti-XIAP, anti-Cl-caspase-3, or anti-GAPDH primary antibody (Cell Signaling Technology), and then with anti-mouse or anti-rabbit secondary antibodies (Sigma-Aldrich), as appropriate. Immunoreactive proteins were visualized using enhanced chemiluminescence (ECL) chemical reagents (Bio-Rad) and ChemiDoc (Bio-Rad), and were analyzed using ImageJ software.

Data Analysis

All data are presented as means±standard error of the mean (SEM). The statistical analysis was determined by Student's t-test. P values of less than 0.05 were considered statistically significant.

### Example 1: Design of Trimer TRAIL-Expressed Ferritin Nanocage

In order to develop a nature-mimetic delivery platform for providing a stable homologous trimer of recombinant TRAIL, the present inventors used ferritin heavy chain nanocages as a scaffold for structure-based design of trivalent ligands. Human ferritin heavy chains self-assemble into a constant 24-subunit structure and form a spherical cage-like architecture. Nanocages not only have the desired physical properties, but the surface can be manipulated to obtain specificity by active proteins or small molecules through simple genetic and chemical modification (Jutz et al., Chem. Rev. 115: 1653-1701, 2015).

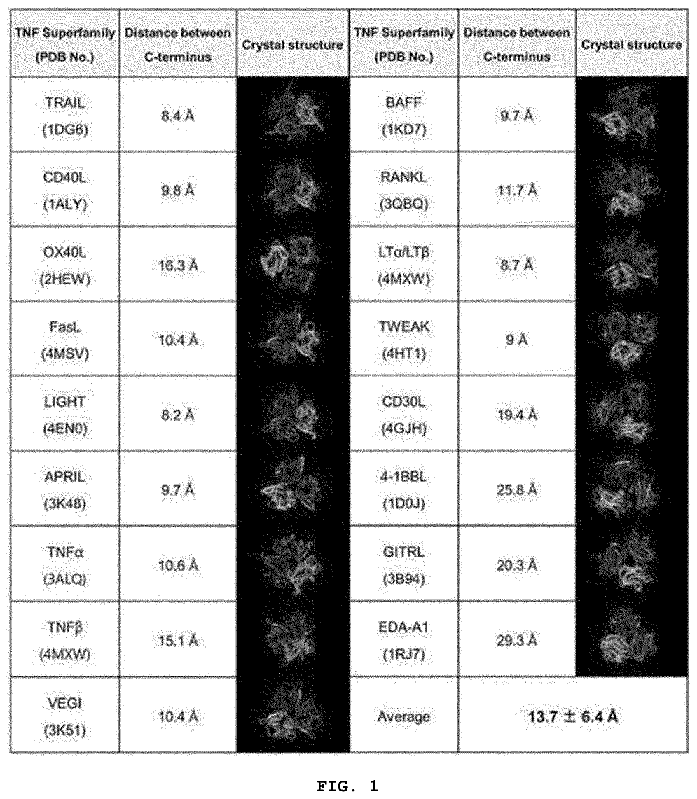

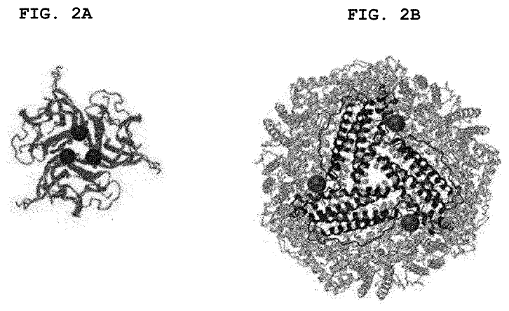

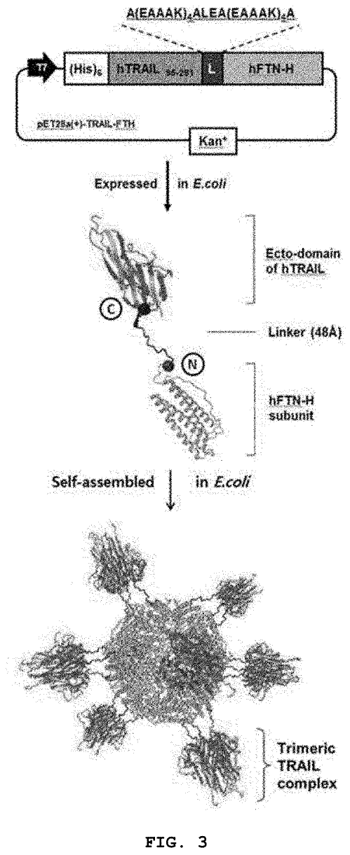

Over the past 20 years, the application potential of ferritin nanocages in drug and vaccine delivery, diagnostics, and biomineralization scaffolds has been extensively evaluated. Based on the crystal structure analysis, given the 4-3-2 axisymmetric structure of the ferritin nanocage, the N-terminus of the nanocage is gathered in a threefold axis and exposed to the outer surface of the shell. Accordingly, the present inventors investigated the presentation of trimeric TRAIL in ferritin nanocages by structural combination based on the analysis of the three-dimensional structure. First, it was determined how the trimeric TRAIL could be presented as native-like conformations around the triple axis on the surface of the ferritin nanocage. When the distance between the ferritin N-terminus (Asp₅) of the triple axis is 28 Å and the distance between the TRAIL foreign domain C-terminus (Leu₂₂₈) of the triple axis is 8.4 Å (FIGS. 1 and 2A-2B), the trimer TRAIL C-terminus cannot coincide with the N-terminus of the ferritin subunit around each triaxial on the nanocage surface. Thus, a linker with rigid and flexible sectors was designed to compensate for the distance between the ferritin N-terminus and the TRAIL C-terminus and form a geometry consistent with the TRAIL homo-trimer on the nanocage surface. As shown in FIGS. 2A-2B and 3, the extracellular-domain of TRAIL was genetically fused to the human ferritin heavy chain by adding a linker. Three of the N-terminal-fused TRAILs on the triple axis of ferritin form a trimer-like structure on the surface of the ferritin nanocage. As the 24 monomeric ferritin subunits are self-assembled into a cage structure, a total of 8 natural-like TRAIL homo-trimers can be displayed on the surface of the ferritin nanocage. In general, other members of the TNF superfamily have similar structures and distances between each C-terminus (FIG. 1). Thus, using a similar approach, ferritin nanocages with 4-3-2 axial symmetry can be used as scaffolds to display other members of the TNF superfamily ligand.

### Example 2: Biosynthesis and Physicochemical Properties of TTPN

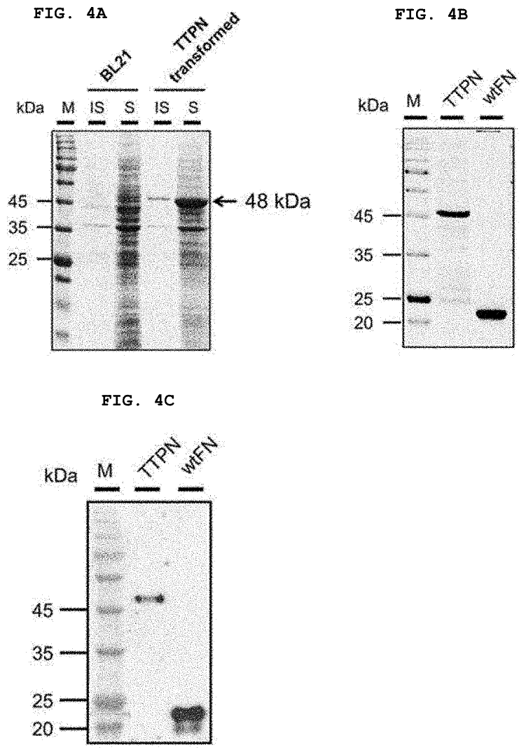

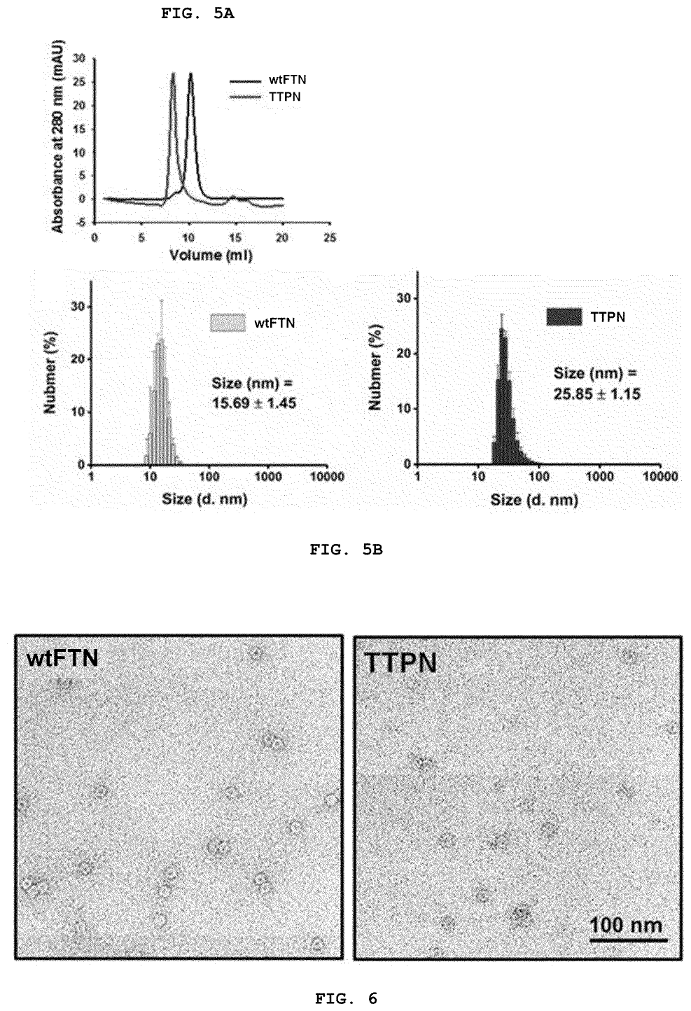

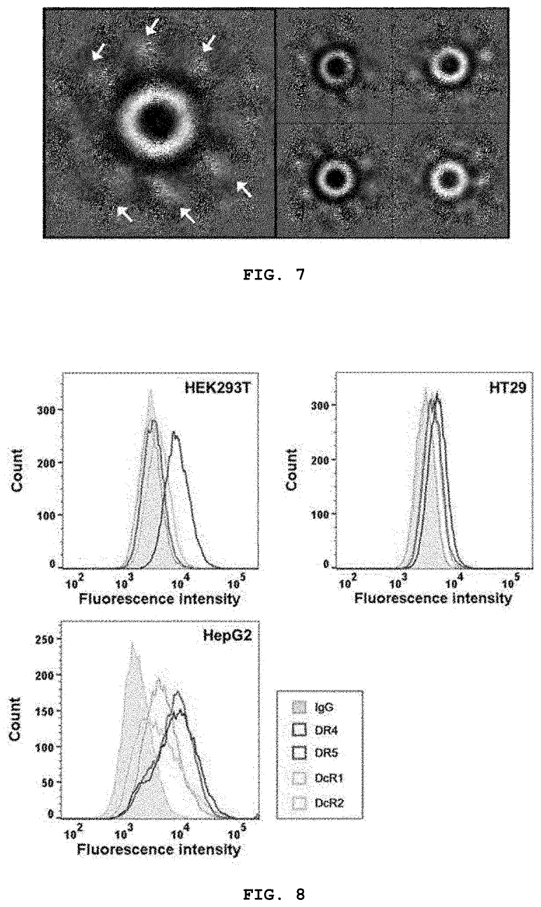

The present inventors determined that the designed TTPN (Trimeric TRAIL-Presenting Nanocage) was successfully expressed as a soluble recombinant protein in E. coli through SDS-PAGE and Western blot analysis (FIG. 4A-4C). Self-assembly of TTPN was evaluated through size exclusion chromatography and dynamic light scattering analysis (DLS) through high-speed protein liquid chromatography (FPLC, FIG. 5A-5B). The size exclusion chromatography of TTPN showed a prominent peak in the elution profile, indicating that the nanocage was well formed. As shown in FIG. 5B, the TTPN formed as described above is slightly larger than the wild type ferritin nanocage (wtFTN). TTPN forms nano-sized particles having an average size of 25.85 nm measured by dynamic light scattering (DLS) analysis. In addition, the properties of TTPN were also confirmed by transmission electron microscopy (TEM) images (FIGS. 6 and 7). The TTPN had a uniform spherical nano-sized particle structure with an average size of 24-28 nm, which is slightly larger than wtFTN. On the other hand, using negative dye transmission electron microscopy to observe the morphology more clearly, TTPN clearly showed visible spikes protruding from the spherical core, whereas wtFTN showed smooth spherical particles. As a result of performing a two-dimensional class analysis on a TEM image by randomly selecting a single particle, it was found that the spikes were distributed in an average of 4 to 6 arms on the surface of the nanocage, suggesting that TRAIL trimer spikes were formed and decorated the surface of the nanocage. Based on the above data, the present inventors have succeeded in designing and generating a TTPN that presents a trimeric TRAIL-like complex in a natural structure on a self-assembled nanocage as a symmetric structure.

### Example 3: Binding Kinetics, Affinity and Stability of TTPN

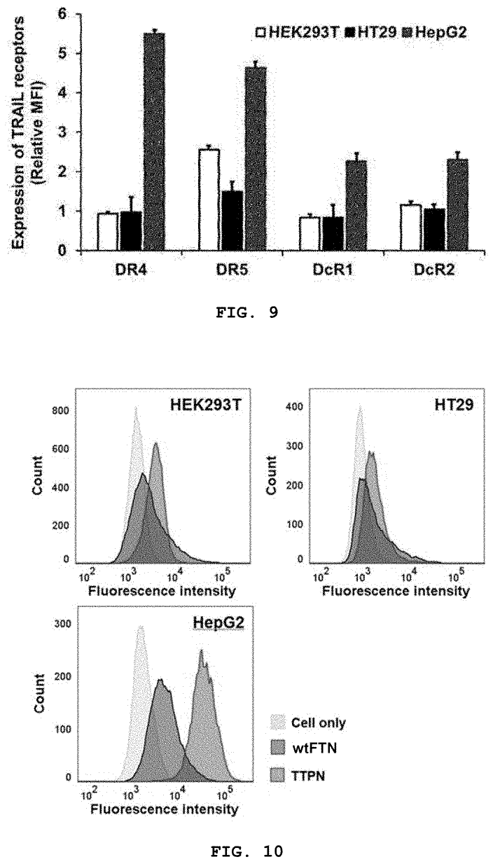

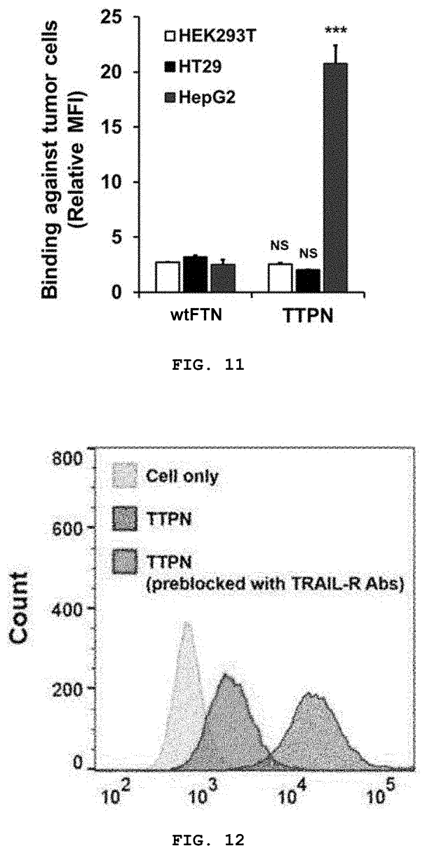

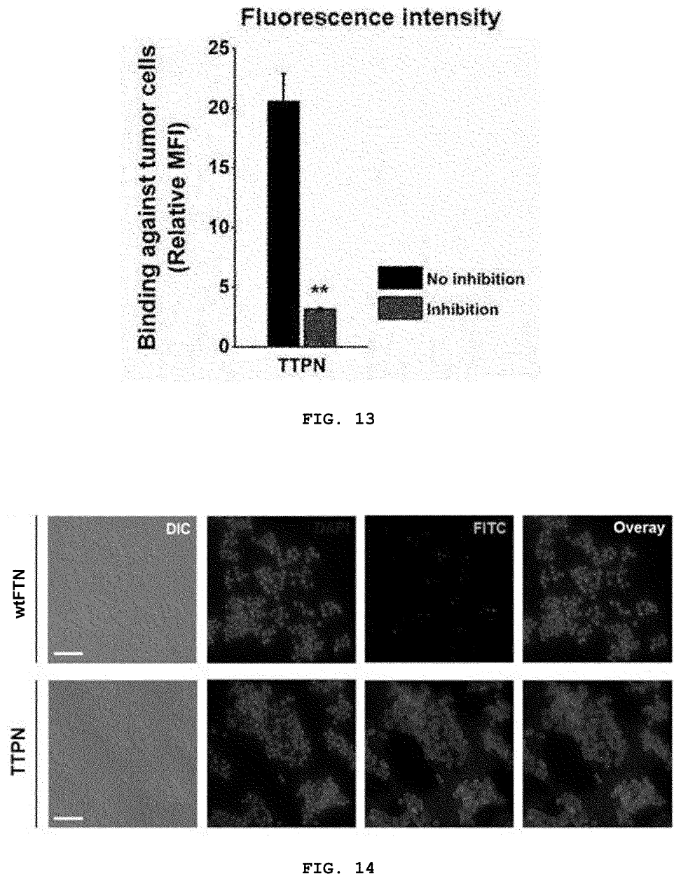

To confirm whether TTPN targets the TRAIL receptor on the surface of tumor cells, the binding ability of TTPN in HepG2 hepatocellular carcinoma, HT29 colon carcinoma and HEK293T cells was evaluated in vitro. HepG2 cells are known to express a greater amount of DR4/DR5 than DcR1/DcR2. As a result of actual analysis, the expression levels of DR4/DR5 and DcR1/DcR2 were nearly 5.46/4.63 and 2.26/2.31 fold, respectively, relative to the IgG control (FIGS. 8 and 9). As a control, analysis results for HT29 cells and HEK293T cells known to be resistant to TRAIL show low levels of DR4/DR5 expression as shown in FIGS. 10 and 11. As shown in FIGS. 10 and 11, TTPN had a greater effect than wtFTN in binding on the surface of HepG2 cells. Since HepG2 cells had high expression of DR4 and DR5, the target specificity of TTPN was higher in HepG2 cells than in HT29 and HEK293T cells. In addition, considering that the binding of TTPN is reduced by pre-incubation with four anti-TRAIL receptor antibodies, TTPN specifically binds to TRAIL receptors on the surface of tumor cells (FIGS. 12 to 14).

In addition, in order to confirm the binding kinetics and affinity of TTPN, DR4 and DR5 immobilized on a sensor chip through protein A and Fc domains were used to compare with the monomeric TRAIL (mTRAIL) extracellular domain. Surface plasmon resonance (SPR) analysis was performed (FIG. 31). As shown in FIG. 31, mTRAIL binds DR4 and DR5 with low affinity as expected, whereas TTPN binds to both receptors with sub-nanomolar affinities. The K_{D} value of TTPN significantly decreased by 330 times compared to DR4 and 37 times compared to DR5 compared to mTRAIL (see Tables 2 and 3). In both receptors, higher binding and lower dissociation rates were observed than mTRAIL, suggesting that the cluster structure of TRAIL, which is well formed on the surface of TTPN, is easily recognized by its receptors, very similar to the homo-trimeric structure in nature.

TABLE 2
Summary of Surface Plasmon Resonance (SPR) Assays for Affinity
and Kinetics of TTPN Binding to Immobilized DR4

| | DR4-Fc | | |
|---|---|---|---|
| | ka (M⁻¹ · S⁻¹) | kd (s⁻¹) | K_{D} (M) |
| mTRAIL | 8.68 (±7.12) · 10² | 5.27 (±2.14) · 10⁻⁵ | 2.47 (±2.27) · 10⁻⁷ |
| TTPN | 3.23 (±0.26) · 10⁴ | 2.42 (±0.48) · 10⁻⁵ | 7.47 (±1.21) · 10⁻¹⁰ |

TABLE 3
Summary of Surface Plasmon Resonance (SPR) analysis for the
affinity and kinetics of TTPN binding to immobilized DR5

| | DR5-Fc | | |
|---|---|---|---|
| | ka (M⁻¹ · S⁻¹) | kd (s⁻¹) | K_{D} (M) |
| mTRAIL | 1.48 (±0.12) · 10³ | 3.87 (±1.13) · 10⁻⁵ | 2.54 (±0.56) · 10⁻⁸ |
| TTPN | 6.62 (±4.19) · 10⁴ | 1.49 (±1.13) · 10⁻⁵ | 6.82 (±5.72) · 10⁻¹⁰ |

The affinity K_{D} was determined from the formula of K_{D}=kd/ka. Results are based on representative sensorgrams obtained from saturation binding reactions averaged over at least three independent runs of SPR measurements (FIG. 31).

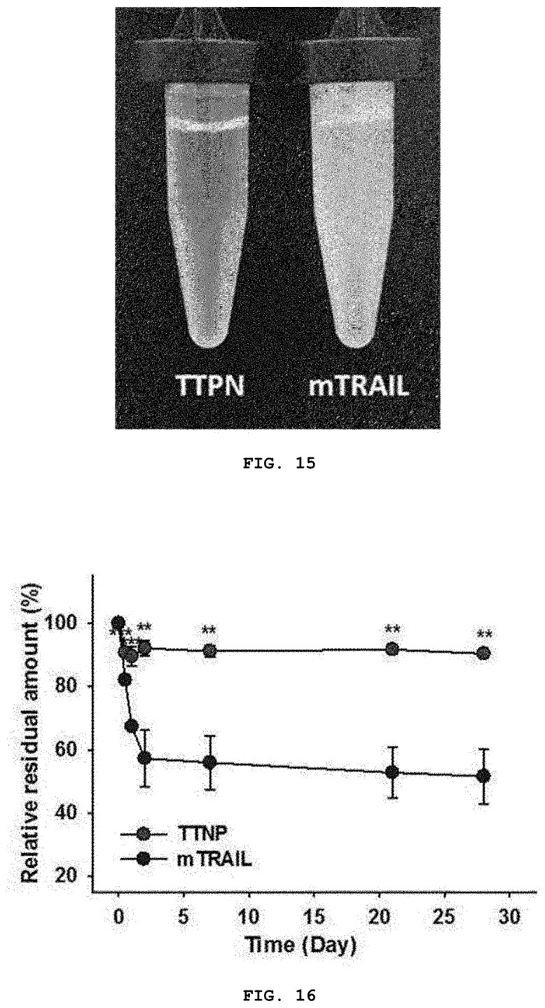

In addition, the present inventors also investigated the in vitro stability of TTPN, because many TRAIL variants developed previously showed liver toxicity problems and instability in solution and rapid aggregation at high concentration in clinical studies according to previous reports, thus, limiting the dosage. However, in the present invention, surprisingly, as shown in FIG. 15, mTRAIL precipitated and aggregated rapidly, while TTPN exhibited remarkably improved stability. In addition, the amount of mTRAIL in the soluble form rapidly dropped to 57% of the initial concentration within 2 days, but more than 90% of the TTPN was still maintained in the soluble form after 1 month (FIG. 16). Overall, the nanocage particle structure of the natural-like trimer TRAIL of the invention substantially improved the recognition ability by improved affinity and stability, which supports the inventors' concept that TTPN according to an embodiment of the present invention can be a promising apoptosis agent for tumor cells.

### Example 4: In Vitro Apoptosis Ability of TTPN

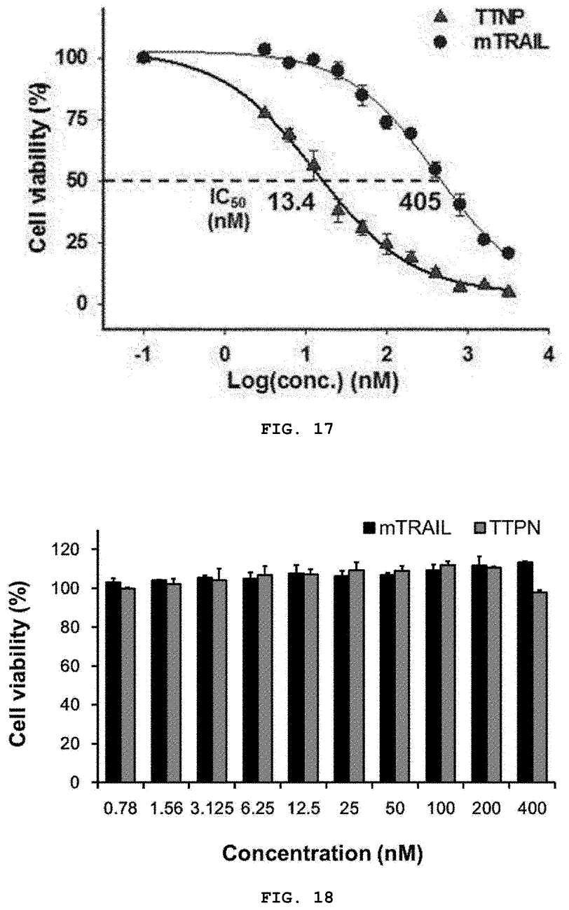

In order to evaluate the TRAIL-mediated apoptosis capacity of TTPN, the present inventors first measured the cell viability of HepG2, HT29 and HEK293T cells against mTRAIL, TTPN and wtFTN as a control. Cells were treated with TTPN, mTRAIL and wtFTN for 24 hours and cell viability was measured using Cell Counting Kit-8 (CCK-8). As shown in FIG. 17, TTPN showed concentration-dependent apoptosis in TRAIL-sensitive HepG2 cells. On the other hand, it is assumed that the low apoptosis rate of HEK293T cells related to TTPN is due to the low levels of TRAIL receptors (DR4 and DR5) expressed in the HEK293T cells (FIG. 18).

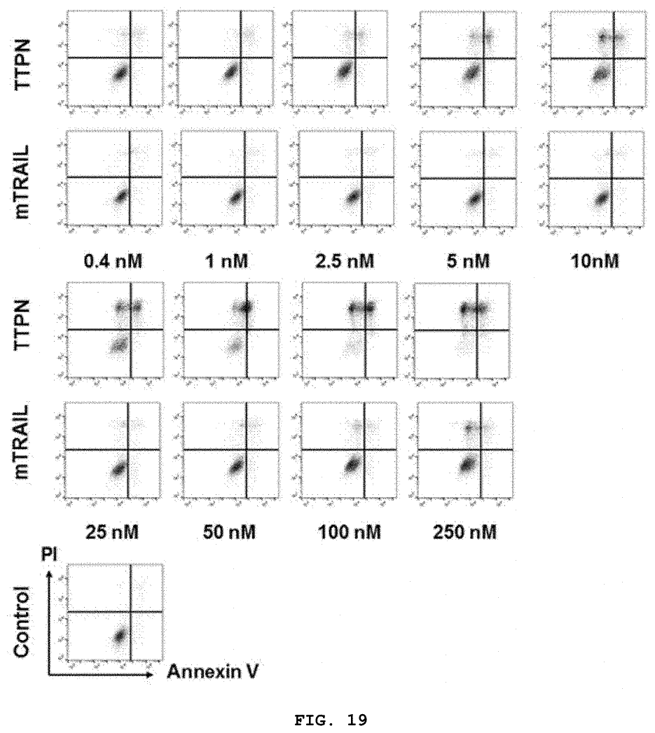

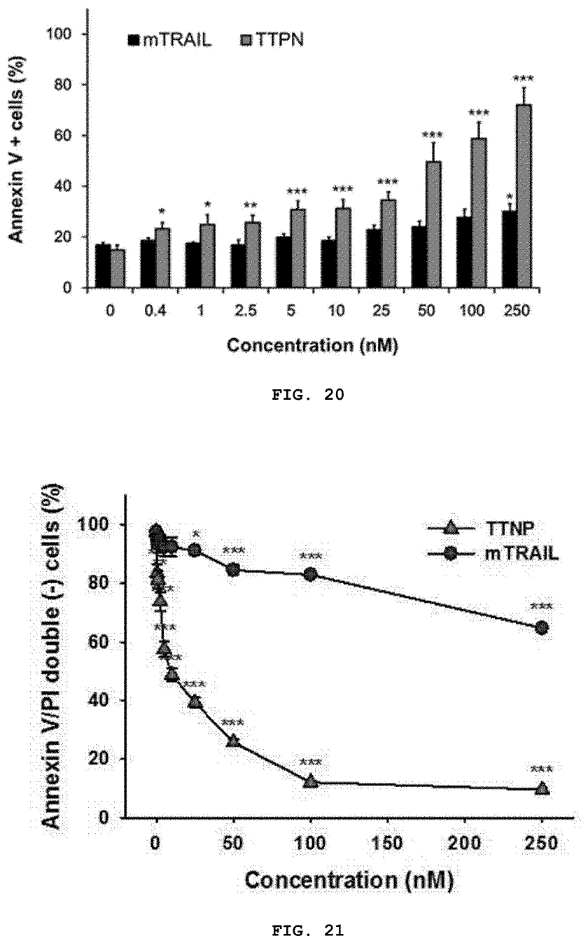

In particular, HepG2 cells reached 50% apoptosis with a low concentration of 13.4 nM TTPN (IC₅₀), whereas the IC₅₀ in mTRAIL-treated cells was 405 nM, which is a concentration 30 times higher than that of TTPN. In addition, to investigate whether apoptosis induced by TTPN is induced by the pro-apoptosis pathway of tumor cells, fluorescence-activated cell sorting (FACS) using double staining of Annexin V/propidium iodide (PI) was performed. Analyzing apoptosis by fluorescence-activated cell sorting) analysis, concentration-dependent apoptosis in HepG2 cells was observed as Annexin V/PI double positive cells (FIG. 19). In addition, Annexin V-positive cells showing initial apoptosis were significantly detected in 0.4 nM TTPN, but no substantial detection of Annexin V-positive cells was observed until treatment with 25 nM mTRAIL (FIG. 20). The percentage (%) of surviving tumor cells for Annexin V/PI double negative signal (PI: marker of late apoptosis and necrosis) of TTPN-treated group was lower than mTRAIL-treated group, significantly [94.3% (not significant) for 0.4 nM mTRAIL, 83.5% (p<0.05) for 0.4 nM TTPN; 12.0% (p<0.001) for 100 nM TTNP and 82.9% (p<0.001) for 100 nM mTRAIL, respectively] (FIG. 21). Thus, the above results suggest that nanocage particles of natural trimer-like TRAIL in TTPN increase the apoptotic effect, which is consistent with the observed increased affinity and stability of TTPN.

### Example 5: Preparation, Physicochemical Characterization, and Stability of DOX-Loaded TTPNs

5-1: Design of DOX-TTPNs

To overcome the resistance of TRAIL-mediated apoptosis, the present inventors applied the additional strategy of using doxorubicin (DOX) as a re-sensitizing agent. A number of studies have demonstrated that radiotherapy and anticancer chemotherapeutics, such as cisplatin, doxorubicin and tunicamycin, when combined with TRAIL monotherapy, can re-sensitize TRAIL-resistant tumor cells in vitro and in vivo (Refaat et al., Oncol. Lett. 7(5): 1327-1332, 2014; Oh et al., J. Control. Release 2015, 220(Pt B): 671-681, 2015; Zinonos et al., Anticancer Res. 34(12): 7007-7020, 2014). Among these chemotherapeutic drugs, DOX acts by regulating TRAIL receptor (i.e., DR5) levels and pro- and anti-apoptotic proteins at points within intrinsic and extrinsic apoptotic pathways; thus, combined treatment with DOX and TRAIL may amplify TRAIL-induced apoptosis (Zinonos et al., Anticancer Res. 34(12): 7007-7020, 2014; Bae et al., Biomaterials 33(5): 1536-1546, 2012). Notably, the present inventors took advantage of the ability of DOX to form a stable complex with a metal cation to create Cu-DOX, which is easily encapsulated into the inner cavity of the ferritin nanocage. Ferritin nanocages are cellular iron storage proteins that allow encapsulation of metal-complexed molecules. These properties give DOX-loaded ferritin nanocages a therapeutic advantage over free drug.

5-2: Preparation and Characterization of DOX-TTPNs

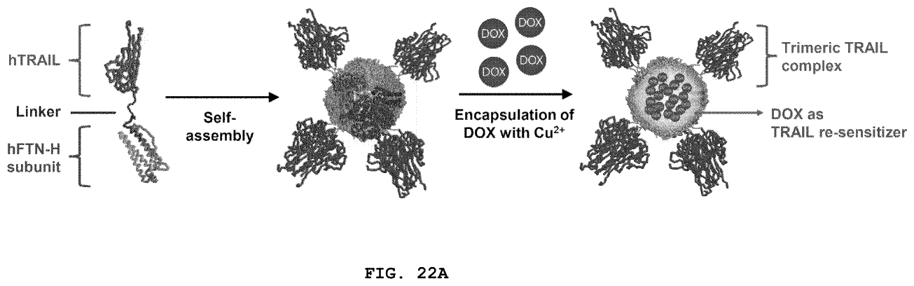

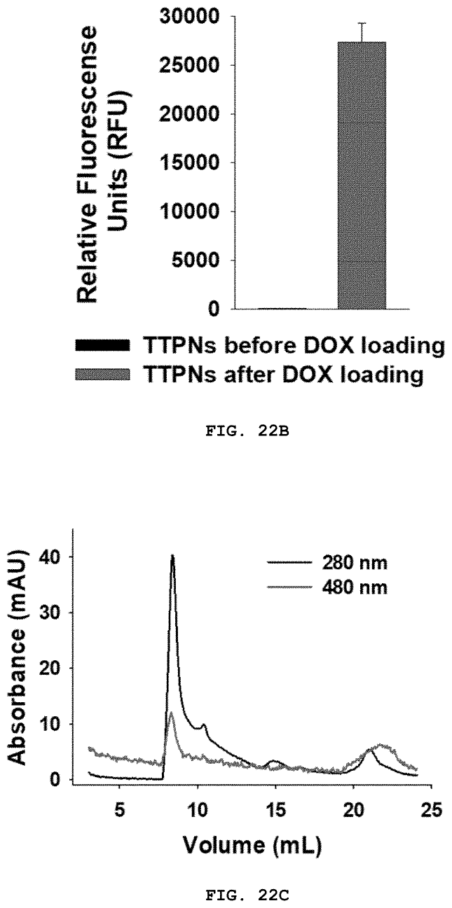

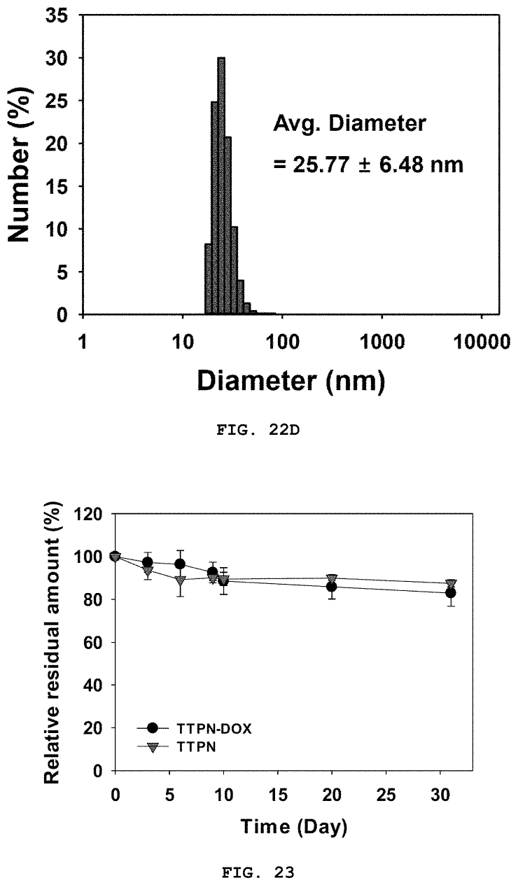

The present inventors demonstrated encapsulation of metal-complexed DOX in TTPNs, termed DOX-TTPNs (FIG. 22A), which represent a further improvement and optimization of ferritin nanocage platforms described in previous studies (Kih et al., Biomaterials 180: 67-77, 2018; Lee et al., Adv. Mater. 30(10): 1705581, 2018). DOX-TTPNs were prepared by pre-complexation of DOX with Cu²⁺ and incubation with TTPNs, followed by removal of free DOX. The loading efficiency of DOX into TTPNs was determined by size-exclusion chromatography (SEC) and measuring the fluorescence intensity of DOX in TTPNs. As shown in FIG. 22B, the elution profile of DOX-TTPNs exhibited two prominent peaks at 280 nm and 480 nm, each with a similar elution time, indicating that DOX is well-encapsulated within TTPNs. DOX-TTPNs showed no significant difference in diameter compared with TTPNs before DOX encapsulation (FIG. 22C). The amount of incorporated DOX was determined to be ˜30±6 molecules per TTPN, whereas wild-type ferritin encapsulates up to 40 molecules of DOX in its inner cavity. Given that a three-fold channel has been proposed as the primary passageway for metal ions in ferritin, fewer molecules of DOX are deposited in TTPNs, which present TRAIL in its three-fold axis, than in wild-type ferritin (Laghaei et al., Proteins 81(6): 1042-1050, 2013). Taken together, these findings suggest that, although the loading efficiency of DOX in TTPNs was slightly reduced compared to wild-type ferritin, the present inventors successfully developed a nanocage therapeutic that not only carries TRAIL in its native-like trimeric complex structure but also delivers DOX to re-sensitize TRAIL-resistant tumor cells.

Next, to verify the stability of DOX-TTPNs, the present inventors monitored their solubility for 1 month. Several TRAIL-based therapeutics have been shown to exhibit low stability and accelerated aggregation at high concentrations, limiting dosing and causing adverse effects such as hepatotoxicity in clinical studies (Soria et al., J. Clin. Oncol. 28(9): 1527-1533, 2010). Importantly, TTPNs showed excellent stability compared with the monomer form of TRAIL; more than 90% stability of TTPN observed over 1 month, whereas almost 50% monomer form of TRAIL exhibited rapid aggregation within 21 days (Kih et al., Biomaterials 180: 67-77, 2018). Consistent with stability of TTPNs, the amount of DOX-TTPNs in soluble form retained more than 90% of their initial value over 1 month, indicating that DOX-TTPNs were remarkably stable (FIG. 23).

### Example 6: In Vivo Apoptosis Ability and Anti-Tumor Effect of TTPN

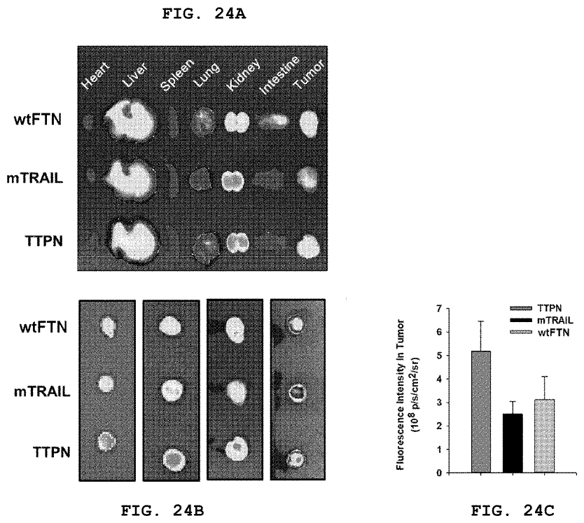

The present inventors investigated the efficacy of TTPN as an anti-tumor agent in HepG2 tumor-bearing mice. Specifically, in order to investigate the delivery efficiency to tumors before observing the anti-tumor effect of TTPN, Cy5.5-labeled TTPN, mTRAIL, and wtFTN were injected intravenously into HepG2 tumor-bearing mice, followed by near-infrared fluorescence (NIRF) imaging. Biodistribution and delivery to tumor tissues were observed. As shown in FIG. 24A-24C, the fluorescence intensity of tumors of mice injected with TTPN was higher than that of wtFTN and mTRAIL. TTPN is more stable than wtFTN and mTRAIL at the tumor site and accumulated more and stayed longer than wtFTN and mTRAIL due to both the interaction with the TRAIL receptor overexpressed in tumor cells and the passive effect through increased permeability and retention (EPR).

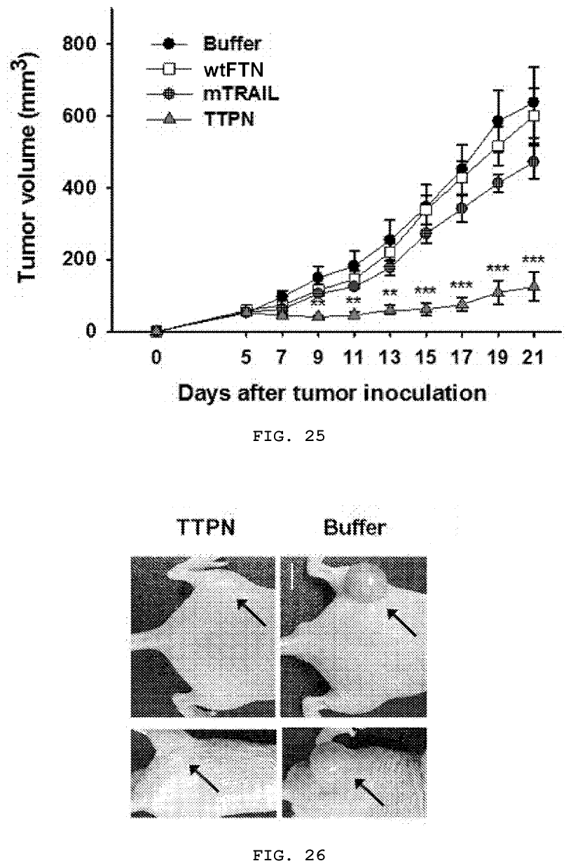

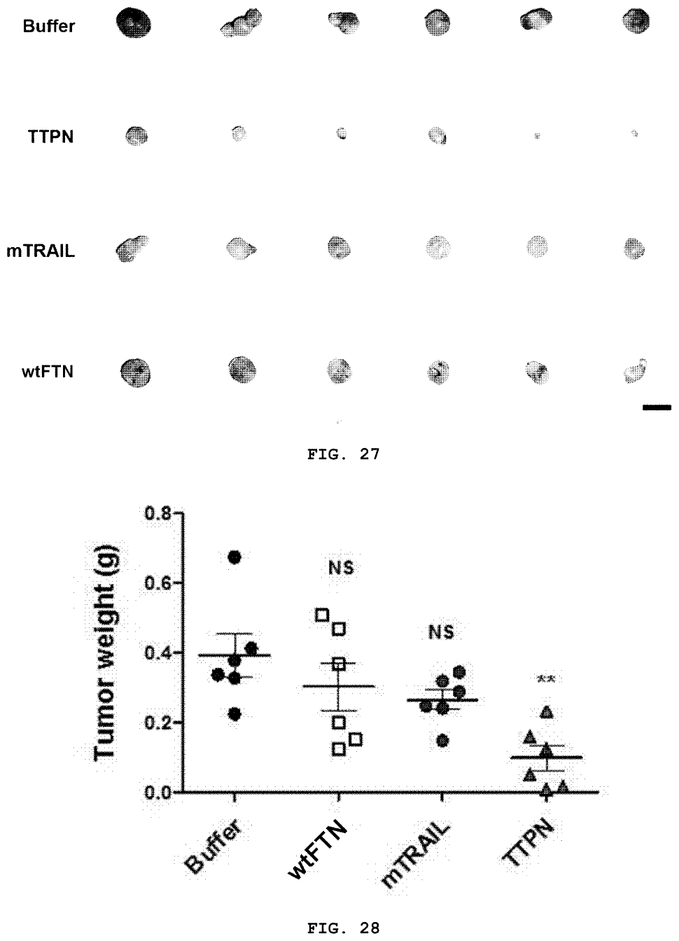

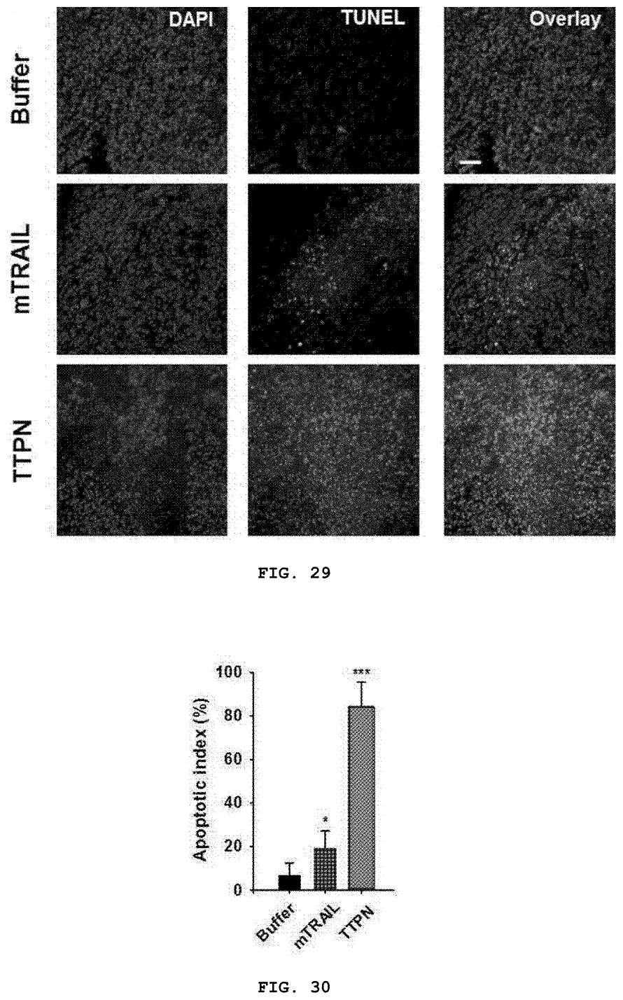

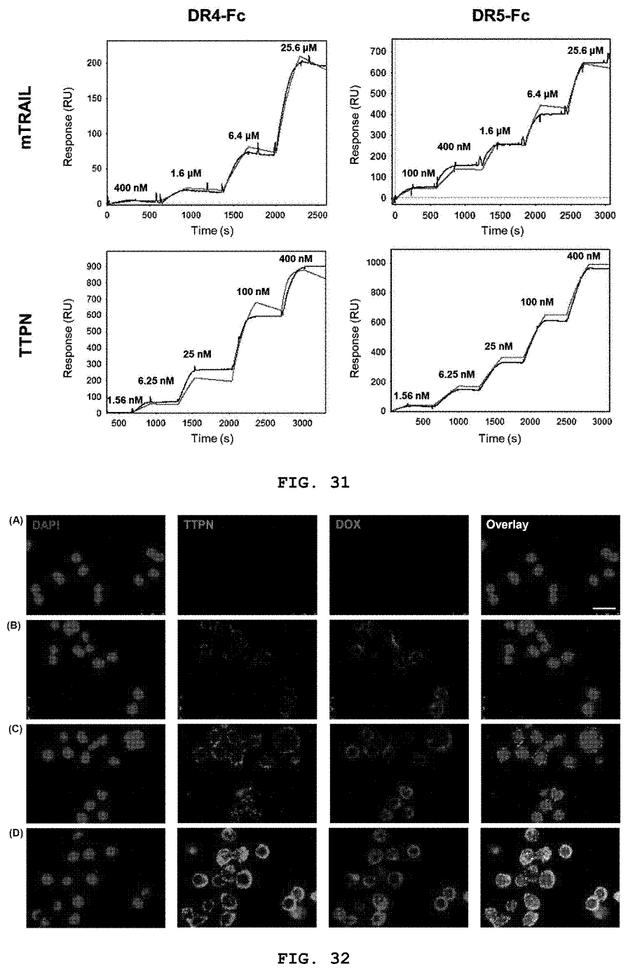

In addition, the tumor growth inhibitory effect of intravenously injected TTPN was evaluated compared with mTRAIL and wtFTN. To this end, HepG2 cells were transplanted into mice as xenografts and the tumor size reached a volume of 80˜100 mm³, and then TTPN (23 mg/kg), mTRAIL (12 mg/kg) or wtFTN (10 mg/kg, corresponding to the molecules of ferritin in the TTPN dose) were administered every 2 days. As shown in FIGS. 25 to 28, tumor growth rate was significantly suppressed in mice injected with TTPN compared to mice injected with other agents tested. TTPN inhibited the tumor volume by 80.52%, which was 3.1 times higher than the effect of a 24-fold molar amount of mTRAIL (25.98% reduction in tumor volume). In addition, to investigate whether tumor growth inhibition by TTPN is induced by apoptosis-inducing activity on tumor cells, tumor tissues from the treated mice were analyzed. On the 15^{th} day after the first injection, mice were euthanized, and apoptosis of tumor tissues was analyzed using terminal deoxynucleotidyl transferase dUTP nick end labeling (TUNEL) staining. TTPN significantly increased apoptosis and TUNEL-positive cells in tumor tissues compared to mTRAIL-treated cells (FIG. 29). In addition, quantification of TUNEL-positive tumor cells treated with TTPN (84.4%) showed a significant increase in the number of apoptotic cells compared to treatment with mTRAIL (18.9%) (FIG. 30). Due to the improved affinity of TRAIL to the TRAIL receptor, the high affinity, and the high apoptosis ability of TTPN, tumor growth was continuously suppressed through strong induction of tumor cell apoptosis.

### Example 7: In Vitro Intracellular Delivery and Pro-Apoptotic Efficacy of DOX-TTPNs

Activation of DRs by TRAIL often leads to clathrin-dependent endocytosis. In particular, it has been reported that trimeric or multimeric TRAIL accelerates the rate of DR5-mediated internalization of cargo via endocytosis by ˜2-fold over 2 hours. TTPNs, which mimic the naturally occurring TRAIL homo-trimeric structure, are readily recognized by TRAIL receptors, as evidenced by their up to 330-fold increased affinity for DR4 and DR5 compared with monomeric TRAIL. The present inventors thus hypothesized that DOX-TTPNs would provide efficient DR5-mediated intracellular delivery of re-sensitizing drugs by virtue of the native-like trimeric TRAIL structure on the nanocage surface, thereby exerting a synergistic effect in re-sensitized TRAIL-resistant cells.

To test this hypothesis, the present inventors first investigated the intracellular delivery of DOX in DOX-TTPNs by analyzing the time-course of DOX-TTPNs internalization within HT29 cells. HT29 cells were incubated with Cy5.5 surface-labeled DOX-TTPNs and then analyzed by fluorescence microscopy. As shown in FIG. 32, DOX-TTPNs bound to the membrane of HT29 cells, and then were distributed to both cytoplasm and membranes. Specifically, after binding of TTPNs to HT29 cells, DOX was localized to the cell membrane at an early time point, and then was rapidly released and localized in the cytoplasm and nuclei. These data indicate that rapid binding of DOX-TTPNs to HT29 cells via DRs leads to endocytosis of DOX-TTPNs followed by intracellular DOX release, suggesting the potential of DOX-TTPNs to re-sensitize cells to TRAIL-induced apoptosis.

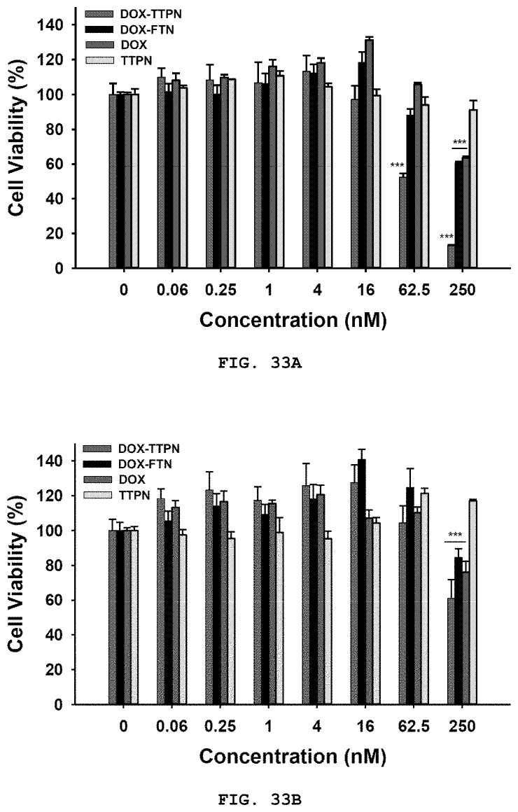

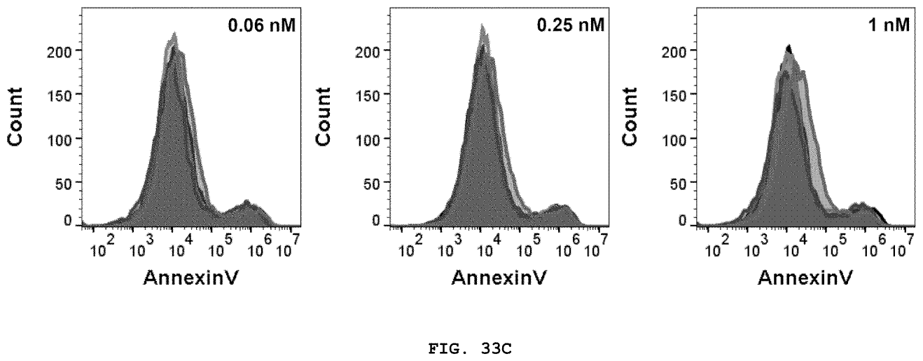

To demonstrate that intracellular DOX released by DOX-TTPNs re-sensitizes TRAIL-resistant cells, the present inventors monitored cell growth in HT29 and MCF7 cells treated with DOX-TTPNs; empty TTPNs, free DOX, and DOX-FTNs (DOX-loaded wild-type ferritin) were used as controls. Cells in each group were incubated for 24 hours, and cell viability was analyzed using a cell counting kit (CCK)-8. Treatment with DOX-TTPNs induced a robust apoptotic response and caused concentration-dependent cell death in both HT29 and MCF7 cells (FIGS. 33A and 33B). Notwithstanding their much lower potency compared with DOX-TTPNs, free DOX and DOX-FTNs also induced cell death at a very high concentration owing to their chemotherapeutic activity. Notably, treatment with 250 nM DOX-TTPNs (DOX concentration, 3.5 μg/ml) caused 87% cell death in HT29 cells compared with controls, whereas the same dose of free DOX and DOX-FTNs caused 36% and 39% cell death relative to controls, respectively. Additionally, because MCF7 cells express lower levels of death receptors, as noted above, and are relatively more resistant to TRAIL than HT29 cells (Zhang et al., Mol. Cancer Res. 6(12): 1861-1871, 2008), a higher concentration of therapeutics (250 nM; p<0.001) was required to induce a similar percentage (40%) of cell death relative to controls compared with HT29 cells (48% of controls at 62.5 nM; p<0.001).

An analysis of cell apoptosis using annexin V staining with fluorescence-activated cell sorting (FACS) revealed synergy between TTPN and DOX. Annexin V is an early apoptosis marker that detects and binds externalized phosphatidylserine, an early event in the apoptotic process, thereby providing a more sensitive indicator of apoptosis than CCK analyses. HT29 cells were treated with low concentrations of DOX-TTPNs, free DOX, and empty TTPNs under the same conditions described above, and annexin V-positive cells were analyzed. As shown in FIG. 33C, the early apoptotic cell profile following treatment with DOX or empty TTPNs essentially mirrored that of untreated cells, indicating that DOX or empty TTPN alone were ineffective in inducing apoptosis. In contrast, treatment with DOX-TTPNs induced early apoptosis at concentrations as low as 0.06 nM (DOX concentration, 8.4 pg/ml). These results indicate that DOX-TTPNs efficiently deliver DOX intracellularly in tumor cells via death receptors, and successfully re-sensitize resistant cells to TRAIL-mediated apoptosis.

### Example 8: Mechanism of DOX-TTPN-Induced Re-Sensitization of TRAIL-Resistant Tumor Cells

To explore the mechanisms underlying the ability of DOX-TTPNs to re-sensitize TRAIL-resistant tumors, the present inventors analyzed expression levels of the TRAIL receptor and several pro- and anti-apoptosis proteins. Previous studies have reported that TRAIL-induced apoptosis is regulated at the TRAIL receptor level, and at points within the extrinsic pathway via activation of death-inducing signaling complex (DISC) and the intrinsic (mitochondrial) pathway through activation of caspase-9 via the release of cytochrome c from mitochondria (Tummers et al., Immunol. Rev. 2017, 277(1): 76-89, 2017; Wang et al., Curr. Pharm. Des. 2014, 20(42): 6714-6122, 2014). Therefore, as indicated above, resistance to TRAIL is achieved through negative regulation of death receptors and death agonists and up-regulation of death antagonists [IAP (inhibitors of apoptosis) family proteins and anti-apoptotic proteins] at extrinsic and intrinsic pathways (Wang et al., Curr. Pharm. Des. 2014, 20(42): 6714-6122, 2014).

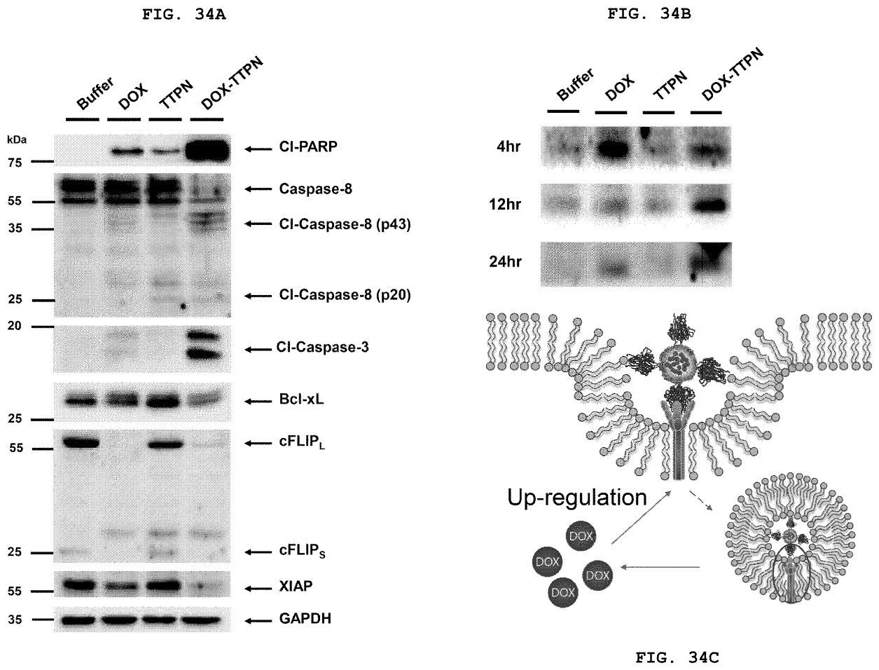

The present inventors selected and analyzed several agonistic and antagonistic proteins for TRAIL-mediated apoptosis, including poly (ADP-ribose) polymerase (PARP), caspase-8, caspase-3, B-cell lymphoma-extra large (Bcl-xL), cellular FLICE (FADD-like IL-1β-converting enzyme)-inhibitory protein (c-FLIP), and X-linked inhibitor of apoptosis protein (XIAP), and the TRAIL receptor DR5, which is important in the regulation of TRAIL resistance in HT29 cells (Zhang et al., Cancer Gene Ther. 12 (3): 228-237, 2005; Saraei et al., Biomed. Pharmacother. 107: 1010-1019, 2018; Geismann et al., Cell Death Dis. 5(10): e1455, 2014]. The levels of these proteins in HT29 cells after treatment with DOX-TTPNs, empty TTPNs, or free DOX were determined by Western blotting. As shown in FIG. 34A, treatment with empty TTPNs caused little or no change in expression levels of this panel of pro-agonistic and antagonistic proteins compared with controls, consistent with cell viability and pro-apoptosis analyses. In the case of treatment with free DOX, all pro-apoptotic proteins showed an increase in expression and all anti-apoptotic proteins showed a decrease in expression. However, the effects of free DOX on these proteins were much smaller than those of DOX-TTPNs. In particular, DOX-TTPNs induced a considerable increase in the cleaved form of caspase-3, which directly induces apoptosis in both extrinsic and intrinsic pathways, and PARP, a substrate of caspase-3. In contrast, both free DOX and empty TTPNs caused little change in caspase-3 or PARP expression. In the latter case, the increases in PARP induced by DOX-2 TTPNs relative to buffer-treated controls (defined as 1) were 4115-, 811- and 457-fold, respectively. The cleaved form of caspase-8, which initiates apoptosis, was also increased in the DOX-TTPN-treated group.

The anti-apoptotic proteins cFLIPL/S, Bcl-xL and XIAP, play specific roles in antagonizing TRAIL-mediated apoptosis, inhibiting cleavage of caspase-8, preventing cytochrome c release upon translocation to the mitochondrial membrane, and suppressing activation of caspase-3 to cleaved form, respectively (Zhang et al., Cancer Gene Ther. 12 (3): 228-237, 2005; Saraei et al., Biomed. Pharmacother. 107: 1010-1019, 2018). Treatment of HT29 cells with DOX-TTPNs decreased expression of each of these anti-apoptotic proteins compared with buffer-treated cells. Importantly, treatment with DOX-TTPNs decreased the levels of XIAP—the most potent caspase inhibitor, which acts by direct binding and inhibiting both initiator and effector 12 caspases—by a remarkable 67-fold compared with buffer treatment; by comparison, treatment with free DOX caused a 34-fold decrease in XIAP levels whereas empty TTPNs had no significant effect.

Given that combined treatment with DOX and TRAIL upregulated the TRAIL receptor, DR5, the present inventors next investigated time-dependent changes in DR5 levels using the above-described analysis (Das et al., Apoptosis 22(10): 1205-1224, 2017). As shown in FIG. 34B, free DOX induced a substantial increase in DR5 expression at 4 hours, although DR5 expression reverted to control-like levels after 12 hours. DOX-TTPN treatment also increased DR5 levels in HT29 cells, but this increase followed a different time course. Specifically, DR5 levels in HT29 cells treated with DOX-TTPNs were lower than those in the free-DOX group at 4 hours, but the increase in DR5 expression levels was greater after 12 hours and was sustained for at least 24 hours. In contrast, empty TTPNs caused no significant change in DR5 expression levels. As indicated above, trimeric or multimeric TRAIL increased the rate of DR5-mediated endocytosis by ˜2-fold over 2 hours and the resulting activation of DRs by TRAIL caused a substantial fraction of DR5 in the cell membrane to shift into the cell. The difference in the kinetics of DR5-downregulation in DOX-TTPN-treated cells compared with free-DOX-treated cells may reflect endocytosis of DR5 with DOX-TTPNs at the early time point (4 hours) (accounting for the relatively smaller increase in DR5 levels), followed by increased expression of DR5 after 12 hours owing to the effects of intracellularly delivered DOX (FIG. 34C). These results demonstrate that, by presenting TRAIL in its native-like trimeric structure and efficiently promoting DR5-mediated intracellular delivery of re-sensitizing drugs, DOX-TTPNs exert a synergistic re-sensitizing effect on TRAIL-resistant cells. Collectively, these results indicate that the mechanisms by which DOX-TTPNs re-sensitize cells to TRAIL-induced apoptosis include efficiently promoting DR5-mediated intracellular delivery of DOX, increasing the levels of DR5 and pro-apoptotic proteins, and decreasing the levels of anti-apoptotic and IAP family proteins.

### Example 9: In Vivo Antitumor Efficacy of DOX-TTPNs

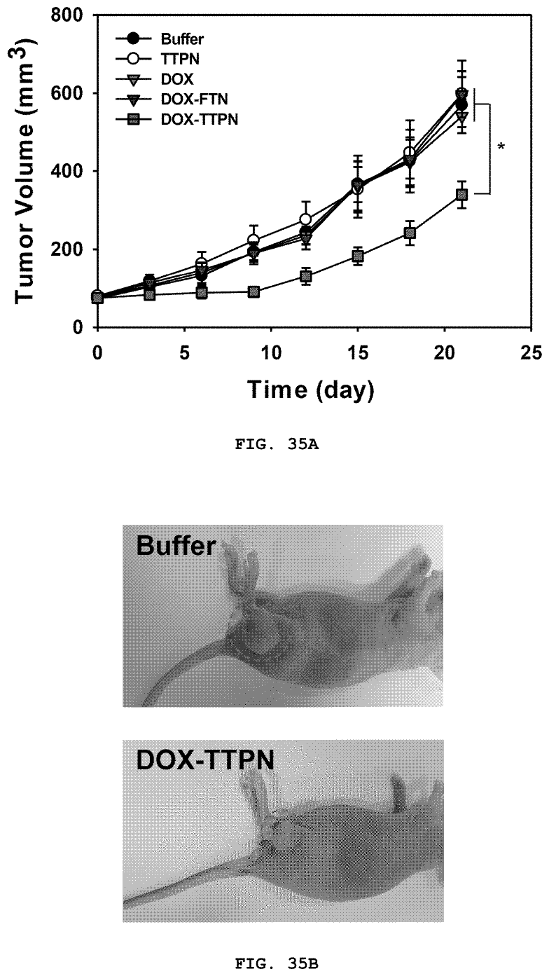

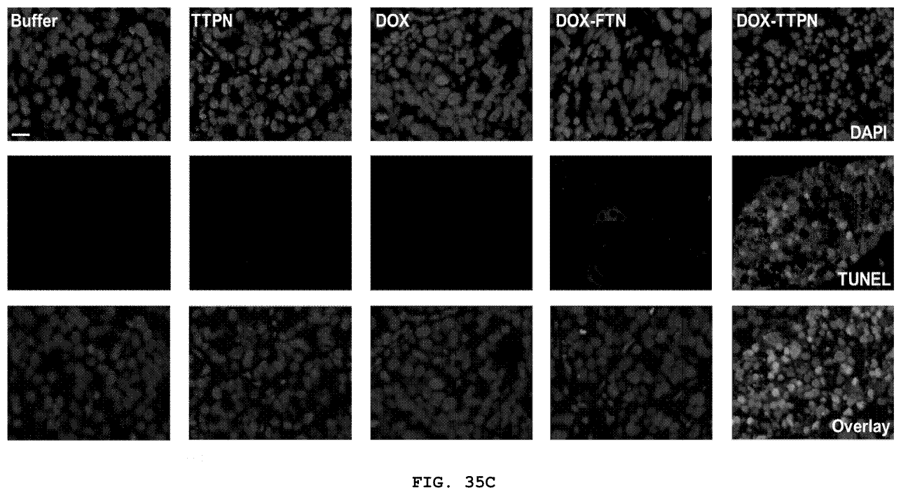

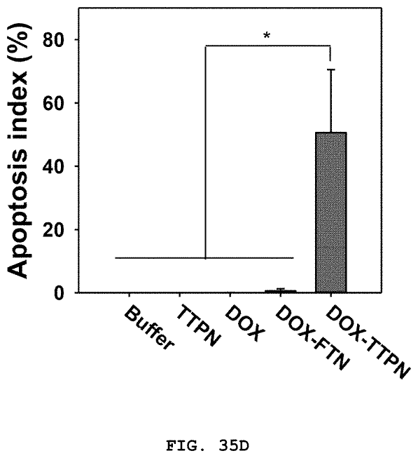

Finally, the present inventors evaluated the apoptotic activity and antitumor efficacy of DOX-TTPNs in a mouse xenograft model. To this end, HT29 cells were implanted in mice, and after tumor volumes reached approximately 80˜100 mm³, mice were intravenously injected with 30 mg/kg of DOX-TTPNs (equivalent to a DOX dose of 0.4 mg/kg), 0.4 mg/kg of DOX, 15 mg/kg of DOX-FTNs (equivalent to the number of moles of ferritin in a dose of TTPNs), or 30 mg/kg of empty TTPNs (equivalent to the number of moles of TRAIL in a dose of TTPNs) five times every 2 days, respectively. As shown in FIGS. 35A and 35B, treatment with DOX-TTPNs successfully suppressed tumor growth, decreasing tumor volumes by 61.5%, whereas free DOX, empty TTPNs, and DOX-FTNs exerted no significant tumor-inhibition effect.

To determine whether tumor growth suppression was induced by re-sensitization to the apoptotic activity of DOX-TTPNs, the present inventors extracted tumor tissue from the above-treated mice at the end of the study and analyzed them for apoptotic cells using TUNEL (terminal deoxynucleotidyl transferase dUTP nick-end labeling) staining. As shown in FIGS. 35C and 35D, treatment with DOX-TTPNs significantly increased the percentage of apoptotic tumor cells in vivo compared with that in mice treated with empty TTPNs, free DOX, or DOX-FTNs; the percentages of TUNEL-positive tumor cells in mice treated with DOX-TTPNs, empty TTPNs, free DOX, or DOX-FTNs were 50.6, 0.01, 0.07 and 0.57%, respectively.

Previous studies have demonstrated success in vitro, and in some cases in vivo, using TRAIL-based combination therapy together with DOX as TRAIL sensitizers (Refaat et al., Oncol. Lett. 7(5), 1327-1332, 2014; Oh et al., J. Control. Release 220(Pt B): 671-681, 2015; Guo et al., J. Control. Release 154(1): 93-102, 2011; Kim et al., Biomater. 34(27): 6444-6453, 2013; Kelly et al., Cancer Biol. Ther. 1(5): 520-533, 2002; Liu et al., Biomater. 33(19): 4907-4916, 2012; Han et al., Biomater. 32(4): 1242-1252, 2011; Hu et al., Oncotarget 7(38): 61832-61844, 2016; Jiang et al., Adv. Funct. Mater. 24(16): 2295-2304, 2014; Jiang et al., Adv. Mater. 27 (6): 1021-1028, 2015; Hu et al., Adv. Mater. 27 (44): 7043-7050, 2015). However, efficient in vivo antitumor efficacy has required relatively high doses of sensitizer (1.5˜7 mg/kg, intravenous administration) that are not clinically practical (Wennerberg et al., Int. J. Cancer 133(7): 1643-1652, 2013). Furthermore, the high concentrations of DOX produce serious cardiac toxicity, which may become even more severe in combination therapy with TRAIL; at doses greater than 1 mg/kg, DOX can exhibit side effects in mice (Chatterjee et al., Cardiol. 115(2): 155-159, 2010). Importantly, the dose of DOX used with TTPNs in this study was 0.4 mg/kg, which is much lower than that in previous studies and is below the cut-off value that causes no side effects. Notably, even at these exceedingly low doses, DOX was capable of re-sensitizing tumor cells to apoptotic activity in a TRAIL-resistant tumor model. The present inventors confirmed that DOX-TTPNs, with their improved stability, apoptotic activity and efficient intracellular delivery, successfully activate TRAIL-mediated apoptosis pathways and inhibit tumor growth in a TRAIL-resistant in vivo model at a minimal dose of the sensitizer, DOX.

The present invention has been described with reference to the above-described examples, but these are merely exemplary, and those of ordinary skill in the art will understand that various modifications and equivalent other embodiments are possible therefrom. Therefore, the true scope of the present invention should be determined by the technical spirit of the

What is claimed is:

1. A fusion protein comprising a tumor necrosis factor (TNF) superfamily protein linked to a self-assembled protein.

2. The fusion protein of claim 1, wherein the self-assembled protein is a small heat shock protein (sHsp), ferritin, vault, P6HRC1-SAPN, M2e-SAPN, MPER-SAPN, or a virus or bacteriophage capsid protein.

3. The fusion protein of claim 1, wherein the self-assembled protein is a ferritin heavy chain protein or a ferritin light chain protein.

4. The fusion protein of claim 1, wherein the self-assembled protein is a ferritin heavy chain protein having the amino acid sequence of SEQ ID NO: 6.

5. The fusion protein of claim 1, wherein the TNF superfamily protein is RAIL, CD40L (CD40 ligand), OX40L (OX40 ligand), FasL (Fas ligand), LIGHT (tumor necrosis factor superfamily member 14), APRIL (A proliferation-inducing ligand), TNF-α (tumor necrosis factor alpha), TNF-β (tumor necrosis factor-beta), VEGI (vascular endothelial growth inhibitor), BAFF (B-cell activating factor), RANKL (receptor activator of nuclear factor kappa-B ligand), LT (lymphotoxin)α/LT (lymphotoxin)β, TWEAK (TNF-related weak inducer of apoptosis), CD30L (CD30 ligand), 4-1BBL (4-1BB ligand), GITRL (glucocorticoid-induced TNF-related ligand), or EDA-A (ectodysplasin A).

6. The fusion protein according to claim 1, wherein the TNF superfamily protein is TRAIL having the amino acid sequence of SEQ ID NO: 2.

7. The fusion protein according to claim 1, wherein the TNF superfamily protein is fused to the N-terminus or C-terminus of the self-assembled protein.

8. The fusion protein according to claim 1, further comprising a linker peptide between the TNF superfamily protein and the self-assembled protein.

9. The fusion protein according to claim 8, wherein the linker peptide has a length of 2 to 50 amino acid residues.

10. The fusion protein according to claim 8, wherein the linker peptide is selected from A(EAAAK)₄ALEA(EAAAK)₄A (SEQ ID NO: 4), (G₄S)ₙ (where n is an integer from 1 to 10), (GS)ₙ (where n is an integer from 1 to 10), (GSSGGS)ₙ (SEQ ID NO: 15, where n is an integer for 1 to 10), KESGSVSSEQLAQFRSLD (SEQ ID NO: 16), EGKSSGSGSESKST (SEQ ID NO: 17), GSAGSAAGSGEF (SEQ ID NO: 18), (EAAAK)ₙ (SEQ ID NO: 19, where n is an integer from 1 to 10), CRRRRRREAEAC (SEQ ID NO: 20), GGGGGGGG (SEQ ID NO: 21), GGGGGG (SEQ ID NO: 22), AEAAAKEAAAAKA (SEQ ID NO: 23), PAPAP (SEQ ID NO: 24), (Ala-Pro)ₙ (where n is an integer from 1 to 10), VSQTSKLTRAETVFPDV (SEQ ID NO: 25), PLGLWA (SEQ ID NO: 26), TRHRQPRGWE (SEQ ID NO: 27), AGNRVRRSVG (SEQ ID NO: 28), RRRRRRRR (SEQ ID NO: 29), and GSSGGSGSSGGSGGGDEADGSRGSQKAGVDE (SEQ ID NO: 30).

11. A protein nanocage produced by self-assembly of the fusion protein according to claim 1.

12. A complex protein nanocage produced by self-assembly of the fusion protein according to claim 1 encapsulating an immunogenic cell death-inducing compound or a TNF superfamily re-sensitizer therein.

13. The complex protein nanocage of claim 12, wherein the immunogenic cell death-inducing compound is encapsulated therein and is an anti-EGFR antibody, a BK channel agonist, bortezomib, the combination of cardiac glycoside and a non-immunogenic apoptosis inducer, cyclophosphamides, the combination of GADD34/PP1 inhibitor and mitomycin, LV-tSMAC, Measles virus, or oxaliplatin.

14. The complex protein nanocage of claim 12, wherein the TNF superfamily re-sensitizer is encapsulated therein and is doxorubicin, cisplatin, gemcitabine, oxaliplatin, irinotecan, camptothecin, celecoxib, curcumin, cinobufotalin, berberine, LY294002, wortmannin, ABT-737, HA14-1, p53 reactivation or induction of massive apoptosis (PRIMA-1).

15. A pharmaceutical composition for treating cancer comprising the protein nanocage according to claim 11 as an active ingredient and at least one pharmaceutically acceptable carrier.

16. The pharmaceutical composition according to claim 15, further comprising at least one immunogenic cell death-inducing compound or a TNF superfamily re-sensitizer.

17. A pharmaceutical composition for treating cancer comprising the complex protein nanocage according to claim 12 as an active ingredient and at least one pharmaceutically acceptable carrier.

18. A method of treating cancer in a subject comprising administering a therapeutically effective amount of the protein nanocage according to claim 11 to the subject.

19. A method of treating cancer in a subject comprising administering a therapeutically effective amount of the complex protein nanocage according to claim 12 to the subject.

20. A method of re-sensitizing TRAIL-resistant tumor cells to TRAIL, comprising treating the tumor cells with the complex protein nanocage according to claim 12.

21. A method of re-sensitizing TRAIL-resistant tumor cells to TRAIL, comprising treating the tumor cells with the protein nanocage of claim 11 and an immunogenic cell death-inducing compound or TNF superfamily re-sensitizer.

22. A method of treating cancer in a subject suffering from TRAIL-resistant cancer, comprising administering a therapeutically effective amount of the complex protein nanocage of claim 12 to the subject.

23. A method of treating cancer in a subject suffering from TRAIL-resistant cancer, comprising administering a therapeutically effective amount of the protein nanocage of claim 11 and an immunogenic cell death-inducing compound or TNF superfamily re-sensitizer to the subject.


SEQUENCE LISTING (30 sequences)

SEQ ID NO: 1 - DNA, 529 bp, Artificial Sequence
  TRAIL 95-281
atgacctctgaggaaaccatttctacagttcaagaaaagcaacaaaatatttctcccctagtgagagaaagaggtcctcagagagtagcagctcacataactgggaccagaggaagaagcaacacattgtcttctccaaactccaagaatgaaaaggctctgggccgcaaaataaactcctgggaatcatcaaggagtgggcattcattcctgagcaacttgcacttgaggaatggtgaactggtcatccatgaaaaagggttttactacatctattcccaaacatactttcgatttcaggaggaaataaaagaaaacacaaagaacgacaaacaaatggtccaatatatttacaaatacacaagttatcctgaccctatattgttgatgaaaagtgctagaaatagttgttggtctaaagatgcagaatatggactctattccatctatcaagggggaatatttgagcttaaggaaaatgacagaatttttgtttctgtaacaaatgagcacttgatagacatggacc

SEQ ID NO: 2 - protein, 188 aa, Artificial Sequence
  TRAIL 95-281
MTSEETISTVQEKQQNISPLVRERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVG

SEQ ID NO: 3 - DNA, 567 bp, Artificial Sequence
  linker peptide
atgacctctgaggaaaccatttctacagttcaagaaaagcaacaaaatatttctcccctagtgagagaaagaggtcctcagagagtagcagctcacataactgggaccagaggaagaagcaacacattgtcttctccaaactccaagaatgaaaaggctctgggccgcaaaataaactcctgggaatcatcaaggagtgggcattcattcctgagcaacttgcacttgaggaatggtgaactggtcatccatgaaaaagggttttactacatctattcccaaacatactttcgatttcaggaggaaataaaagaaaacacaaagaacgacaaacaaatggtccaatatatttacaaatacacaagttatcctgaccctatattgttgatgaaaagtgctagaaatagttgttggtctaaagatgcagaatatggactctattccatctatcaagggggaatatttgagcttaaggaaaatgacagaatttttgtttctgtaacaaatgagcacttgatagacatggaccatgaagccagtttttttggggcctttttagttggctaa

SEQ ID NO: 4 - protein, 46 aa, Artificial Sequence
  linker peptide
AEAAAKEAAAKEAAAKEAAAKALEAEAAAKEAAAKEAAAKEAAAKA

SEQ ID NO: 5 - DNA, 552 bp, Artificial Sequence
  Ferritin
atgacgaccgcgtccacctcgcaggtgcgccagaactaccaccaggactcagaggccgccatcaaccgccagatcaacctggagctctacgcctcctacgtttacctgtccatgtcttactactttgaccgcgatgatgtggctttgaagaactttgccaaatactttcttcaccaatctcatgaggagagggaacatgctgagaaactgatgaagctgcagaaccaacgaggtggccgaatcttccttcaggatatcaagaaaccagactgtgatgactgggagagcgggctgaatgcaatggagtgtgcattacatttggaaaaaaatgtgaatcagtcactactggaactgcacaaactggccactgacaaaaatgacccccatttgtgtgacttcattgagacacattacctgaatgagcaggtgaaagccatcaaagaattgggtgaccacgtgaccaacttgcgcaagatgggagcgcccgaatctggcttggcggaatatctctttgacaagcacaccctgggagacagtgataatgaaagctaa

SEQ ID NO: 6 - protein, 183 aa, Artificial Sequence
  Ferritin
MTTASTSQVRQNYHQDSEAAINRQINLELYASYVYLSMSYYFDRDDVALKNFAKYFLHQSHEEREHAEKLMKLQNQRGGRIFLQDIKKPDCDDWESGLNAMECALHLEKNVNQSLLELHKLATDKNDPHLCDFIETHYLNEQVKAIKELGDHVTNLRKMGAPESGLAEYLFDKHTLGDSDNES

SEQ ID NO: 7 - DNA, 30 bp, Artificial Sequence
  wtFN F
catatgcatcaccatcaccatcacacgacc

SEQ ID NO: 8 - DNA, 24 bp, Artificial Sequence
  wtFN R
aagcttttagctttcattatcact

SEQ ID NO: 9 - DNA, 24 bp, Artificial Sequence
  mTRAIL F
catatgacctctgaggaaaccatt

SEQ ID NO: 10 - DNA, 27 bp, Artificial Sequence
  mTRAIL R
ggatccttagccaactaaaaaggcccc

SEQ ID NO: 11 - DNA, 24 bp, Artificial Sequence
  TTPN 1
catatgacctctgaggaaaccatt

SEQ ID NO: 12 - DNA, 27 bp, Artificial Sequence
  TTPN 2
ctcgagacgaccgcgtccacctcgcag

SEQ ID NO: 13 - DNA, 27 bp, Artificial Sequence
  TTPN 3
ggatccgccaactaaaaaggccccaaa

SEQ ID NO: 14 - DNA, 24 bp, Artificial Sequence
  TTPN 4
aagcttttagctttcattatcact

SEQ ID NO: 15 - protein, 6 aa, Artificial Sequence
  linker peptide
GSSGGS

SEQ ID NO: 16 - protein, 18 aa, Artificial Sequence
  linker peptide
KESGSVSSEQLAQFRSLD

SEQ ID NO: 17 - protein, 14 aa, Artificial Sequence
  linker peptide
EGKSSGSGSESKST

SEQ ID NO: 18 - protein, 12 aa, Artificial Sequence
  linker peptide
GSAGSAAGSGEF

SEQ ID NO: 19 - protein, 5 aa, Artificial Sequence
  linker peptide
EAAAK

SEQ ID NO: 20 - protein, 12 aa, Artificial Sequence
  linker peptide
CRRRRRREAEAC

SEQ ID NO: 21 - protein, 8 aa, Artificial Sequence
  linker peptide
GGGGGGGG

SEQ ID NO: 22 - protein, 6 aa, Artificial Sequence
  linker peptide
GGGGGG

SEQ ID NO: 23 - protein, 13 aa, Artificial Sequence
  linker peptide
AEAAAKEAAAAKA

SEQ ID NO: 24 - protein, 5 aa, Artificial Sequence
  linker peptide
PAPAP

SEQ ID NO: 25 - protein, 17 aa, Artificial Sequence
  linker peptide
VSQTSKLTRAETVFPDV

SEQ ID NO: 26 - protein, 6 aa, Artificial Sequence
  linker peptide
PLGLWA

SEQ ID NO: 27 - protein, 10 aa, Artificial Sequence
  linker peptide
TRHRQPRGWE

SEQ ID NO: 28 - protein, 10 aa, Artificial Sequence
  linker peptide
AGNRVRRSVG

SEQ ID NO: 29 - protein, 8 aa, Artificial Sequence
  linker peptide
RRRRRRRR

SEQ ID NO: 30 - protein, 31 aa, Artificial Sequence
  linker peptide
GSSGGSGSSGGSGGGDEADGSRGSQKAGVDE